(19) United States
(12) Patent Application Publication
(10) Pub. No.: US 2009/0215748 A1
(43) Pub. Date: Aug. 27, 2009

(54) FXR agonists for treating vitamin D associated diseases

(75) Inventor: Douglas Harnish, Pennsburg, PA (US)

Correspondence Address: WYETH/FINNEGAN HENDERSON, LLP, 901 NEW YORK AVENUE, NW, WASHINGTON, DC, 20001-4413, US

(73) Assignee: Wyeth, Madison, NJ (US)

(21) Appl. No.: 12/318,039

(22) Filed: Dec. 19, 2008

Related U.S. Application Data
(60) Provisional application No. 61/008,307, filed on Dec. 20, 2007.

(51) Int. Cl. A61K 31/55; A61P 3/04; A61P 3/10; A61P 3/00; A61P 13/12; A61P 3/14; A61P 5/18; A61P 9/00; A61P 9/10; A61P 9/04; A61P 9/12

(52) U.S. Cl. 514/215

(57) ABSTRACT

Provided are certain methods of treating at least one condition that can be treated by elevating the vitamin D receptor (VDR) activity level in a patient with at least one farnesoid X receptor (FXR) agonist. Also provided are certain methods of modulating levels of Cytochrome P450, family 27, subfamily B, polypeptide 1 (CYP27B1) and 1α,25-dihydroxyvitamin D₃ in cells, certain methods of modulating VDR activity levels, certain methods of modulating levels of an extracellular matrix protein, renin angiotensin system (RAS) pathway, parathyroid hormone, serum creatinine, serum albumin, proteinuria, lipid metabolism, renal lipid deposition, mesangial expansion, glomerulosclerosis, kidney inflammation, blood pressure, bone resorption, and bone formation, certain methods of identifying FXR modulators, certain methods of diagnosing the risk that a patient will develop at least one condition that can be treated by elevating the VDR activity level, and certain methods of characterizing the levels of FXR activity in mammals.

This application claims the benefit of priority to U.S. Provisional Application No. 61/008,307, filed Dec. 20, 2007, the entire contents of which are hereby incorporated herein by reference.

Provided are certain methods of treating at least one condition that can be treated by elevating the vitamin D receptor (VDR) activity level in patients with farnesoid X receptor (FXR) agonisits. Also provided are certain methods of modulating levels of Cytochrome P450, family 27, subfamily B, polypeptide 1 (CYP27B1) and 1α,25-dihydroxyvitamin D₃ in cells, certain methods of modulating VDR activity levels, certain methods of modulating levels of an extracellular matrix protein, renin angiotensin system (RAS) pathway, parathyroid hormone, serum creatinine, serum albumin, proteinuria, lipid metabolism, renal lipid deposition, mesangial expansion, glomerulosclerosis, kidney inflammation, blood pressure, bone resorption, and bone formation, certain methods of identifying FXR modulators, certain methods of diagnosing the risk that a patient will develop at least one condition that can be treated by elevating the VDR activity level, and certain methods of characterizing the levels of FXR activity in mammals.

Nuclear receptors are a superfamily of regulatory proteins that are structurally and functionally related and are receptors for, e.g., steroids, retinoids, vitamin D and thyroid hormones (see, e.g., Evans (1988) Science 240:889-895). These proteins bind to cis-acting elements in the promoters of their target genes and modulate gene expression in response to ligands for the receptors.

Nuclear receptors can be classified based on their DNA binding properties (see, e.g., Evans, supra and Glass (1994) Endocr. Rev. 15:391-407). For example, one class of nuclear receptors includes the glucocorticoid, estrogen, androgen, progestin and mineralocorticoid receptors which bind as homodimers to hormone response elements (HREs) organized as inverted repeats (see, e.g., Glass, supra). A second class of receptors, including those activated by retinoic acid, thyroid hormone, vitamin D₃, fatty acids/peroxisome proliferators (i.e., peroxisome proliferator activated receptor (PPAR)) and ecdysone, bind to HREs as heterodimers with a common partner, the retinoid X receptors (i.e., RXRs, also known as the 9-cis retinoic acid receptors; see, e.g., Levin et al. (1992) Nature 355:359-361 and Heyman et al. (1992) Cell 68:397-406).

RXRs are unique among the nuclear receptors in that they bind DNA as a homodimer and are required as a heterodimeric partner for a number of additional nuclear receptors to bind DNA (see, e.g., Mangelsdorf et al. (1995) Cell 83:841-850). The latter receptors, termed the class II nuclear receptor subfamily, include many which are established or implicated as important regulators of gene expression. There are three RXR genes (see, e.g., Mangelsdorf et al. (1992) Genes Dev. 6:329-344), coding for RXRα, -β, and -γ, all of which are able to heterodimerize with any of the class II receptors, although there appear to be preferences for distinct RXR subtypes by partner receptors in vivo (see, e.g., Chiba et al. (1997) Mol. Cell. Biol. 17:3013-3020). In the adult liver, RXRα is the most abundant of the three RXRs (see, e.g., Mangelsdorf et al. (1992) Genes Dev. 6:329-344), suggesting that it might have a prominent role in hepatic functions that involve regulation by class II nuclear receptors. See also, Wan et al. (2000)Mol. Cell. Biol 20:4436-4444.

The farnesoid X receptor (originally isolated as RIP14 (retinoid X receptor-interacting protein-14), see, e.g., Seol et al. (1995) Mol. Endocrinol. 9:72-85) is a member of the nuclear hormone receptor superfamily and is expressed in the liver, kidney and intestine, among other locations. It functions as a heterodimer with the retinoid X receptor (RXR) and binds to response elements in the promoters of target genes to regulate gene transcription. The farnesoid X receptor-RXR heterodimer binds with highest affinity to an inverted repeat-1 (IR-1) response element, in which consensus receptor-binding hexamers are separated by one nucleotide. The farnesoid X receptor is part of an interrelated process, in that the receptor is activated by bile acids (the end product of cholesterol metabolism) (see, e.g., Makishima et al. (1999) Science 284:1362-1365, Parks et al. (1999) Science 284:1365-1368, Wang et al. (1999) Mol. Cell. 3:543-553), which serve to inhibit cholesterol catabolism. See also, Urizar et al. (2000) J. Biol. Chem. 275:39313-39317. The activity of farnesoid X receptor has been implicated in physiological processes including but not limited to triglyceride metabolism, catabolism, transport or absorption, bile acid metabolism, catabolism, transport or absorption, re-absorption or bile pool composition, and cholesterol metabolism, catabolism, transport, absorption or reabsorption.

Nuclear receptor activity, including the farnesoid X receptor activity, has been implicated in a variety of diseases and disorders, including, but not limited to, hyperlipidemia and hypercholesterolemia, and complications thereof, including without limitation coronary artery disease, angina pectoris, carotid artery disease, strokes, cerebral arteriosclerosis and xanthoma, (see, e.g., International Patent Application Publication No. WO 00/57915), hyperlipoproteinemia (see, e.g., International Patent Application Publication No. WO 01/60818), hypertriglyceridemia, lipodystrophy, peripheral occlusive disease, ischemic stroke, hyperglycemia and diabetes mellitus (see, e.g., International Patent Application Publication No. WO 01/82917), disorders related to insulin resistance including the cluster of disease states, conditions or disorders that make up “metabolic syndrome” or “Syndrome X” such as glucose intolerance, an increase in plasma triglyceride and a decrease in high-density lipoprotein cholesterol concentrations, hypertension, hyperuricemia, smaller denser low-density lipoprotein particles, and higher circulating levels of plasminogen activator inhibitor-1, atherosclerosis and gallstones (see, e.g., International Patent Application Publication No. WO 00/37077), disorders of the skin and mucous membranes (see, e.g., U.S. Pat. Nos. 6,184,215 and 6,187,814, and International Patent Application Publication No. WO 98/32444), obesity, acne (see, e.g., International Patent Application Publication No. WO 00/49992), and cancer, cholestasis, Parkinson's disease and Alzheimer's disease (see, e.g., International Patent Application Publication No. WO 00/17334).

Vitamin D is a steroid hormone involved in the regulation of mineral metabolism and bone growth. Sources of vitamin D include exposure to sunlight, diet, and dietary supplements. Vitamin D exists in multiple forms. Dietary sources of vitamin D often include a form of vitamin D called ergocalciferol or vitamin D2. Dermal synthesis yields a form of vitamin D called cholecalciferol or vitamin D3. Vitamin D2 and vitamin D3 can bind to vitamin D-binding protein and are transported to the liver, where they undergo hydroxylation, producing low activity forms of vitamin D, including 25 hydroxyvitamin D₃ (25(OH)D₃). 25 Hydroxyvitamin D₃ is converted to a highly active form of vitamin D, 1α,25-dihydroxyvitamin D₃ (1,25(OH)₂D₃), by the enzyme 25-hydroxyvitamin D₃ 1α-hydroxylase, encoded by the Cytochrome P450, family 27, subfamily B, polypeptide 1 (CYP27B1) gene. The active forms of vitamin D can bind to vitamin D receptors, which are nuclear receptors involved in regulation of gene expression, and thus, modulate gene expression of VDR target genes.

Vitamin D deficiency has been associated with conditions including chronic kidney disease, cardiovascular disease, and bone disease. Some studies have suggested that increasing levels of vitamin D can be protective in chronic kidney disease. Vitamin D receptor ligands have also been pursued for the treatment of bone diseases including osteoporosis. Mice deficient in VDR or CYP27B1 show defects in bone formation and also display cardiovascular effects such as increased blood pressure and left ventricular hypertrophy. Clinical studies have also revealed inverse relationships between vitamin D levels and renin activity and blood pressure in patients with essential hypertension.

Currently, vitamin D or synthetic vitamin D receptor ligands may be administered to patients to elevate levels of VDR activity or to mitigate the detrimental effects of vitamin D deficiency. These treatment strategies are limited by side effects including hypercalcemia and hypercalcinuria. In part, these side effects are caused by vitamin D-VDR mediated increases in intestinal calcium absorption. Additional treatments for vitamin D deficiency and diseases associated with vitamin D receptor levels are needed. Effective and safe treatments for vitamin D deficiency and diseases associated with vitamin D receptor levels, with fewer side effects, are also needed.

Provided are methods of treating at least one condition that can be treated by elevating the vitamin D receptor (VDR) activity level in a patient. The methods include administering to the patient a therapeutically effective amount of at least one farnesoid X receptor (FXR) agonist. In some embodiments, the at least one FXR agonist elevates the level of Cytochrome P450, family 27, subfamily B, polypeptide 1 (CYP27B1), to thereby elevate the level of VDR activity in the patient.

Also provided are methods of modulating the level of at least one of CYP27B1 and 1α,25-dihydroxyvitamin D₃ in a cell. The methods include providing an effective amount of at least one FXR modulator, to thereby modulate the level of at least one of CYP27B1 and 1α,25-dihydroxyvitamin D₃ in the cell.

Also provided are methods of modulating the VDR activity level in a patient. The methods include administering to the patient an effective amount of at least one FXR modulator, to thereby modulate the VDR activity level in the patient.

Also provided are methods of modulating the level of at least one of an extracellular matrix protein, RAS pathway, parathyroid hormone, serum creatinine, serum albumin, proteinuria, lipid metabolism, renal lipid deposition, mesangial expansion, glomerulosclerosis, kidney inflammation, blood pressure, bone resorption, and bone formation in a patient. The methods include administering to the patient an effective amount of at least one FXR modulator. In some embodiments, the FXR modulator modulates the level of CYP27B1 in the patient, to thereby modulate the level of at least one of an extracellular matrix protein, RAS pathway, parathyroid hormone, serum creatinine, serum albumin, proteinuria, lipid metabolism, renal lipid deposition, mesangial expansion, glomerulosclerosis, kidney inflammation, blood pressure, bone resorption, and bone formation in the patient.

Also provided are methods of identifying a FXR modulator. The methods include providing a test agent to a cell; determining the level of at least one of CYP27B1 and 1α,25-dihydroxyvitamin D₃ in the cell; and selecting a FXR modulator which modulates the level of at least one of CYP27B1 and 1α,25-dihydroxyvitamin D₃ in the cell.

Also provided are further methods of identifying a FXR modulator. The methods include administering a test agent to a patient; determining the VDR activity level in the patient; and selecting a FXR modulator which modulates the VDR activity level in the patient.

Also provided are further methods of identifying a FXR modulator. The methods include administering a test agent to a patient; determining the level of at least one of the following in the presence and/or absence of the test agent: (a) an extracellular matrix protein, (b) RAS pathway, (c) parathyroid hormone, (d) serum creatinine, (e) serum albumin, (f) proteinuria, (g) lipid metabolism, (h) renal lipid deposition, (i) mesangial expansion, (j) glomerulosclerosis, (k) kidney inflammation, (l) blood pressure, (m) bone resorption, and (n) bone formation in the patient; and selecting a FXR modulator which modulates the level of at least one of: (a) an extracellular matrix protein, (b) RAS pathway, (c) parathyroid hormone, (d) serum creatinine, (e) serum albumin, (f) proteinuria, (g) lipid metabolism, (h) renal lipid deposition, (i) mesangial expansion, (j) glomerulosclerosis, (k) kidney inflammation, (l) blood pressure, (m) bone resorption, and (n) bone formation in the patient.

Also provided are further methods of treating at least one condition that can be treated by elevating the VDR activity level in a patient. The methods include administering to the patient a therapeutically effective amount of at least one FXR agonist. In some embodiments, the at least one FXR agonist is identified by providing a test agent to a cell; determining the level of at least one of CYP27B1 and 1α,25-dihydroxyvitamin D₃ in the cell; and selecting a FXR agonist which elevates the level of at least one of CYP27B1 and 1α,25-dihydroxyvitamin D₃ in the cell.

Also provided are further methods of treating at least one condition that can be treated by elevating the VDR activity level in a patient. The methods include administering to the patient a therapeutically effective amount of at least one FXR agonist. In some embodiments, the at least one FXR agonist is identified by administering a test agent to a patient; determining the VDR activity level in the patient; and selecting a FXR agonist which elevates the VDR activity level in the patient.

Also provided are further methods of treating at least one condition that can be treated by elevating the VDR activity level in a patient. The methods include administering to the patient a therapeutically effective amount of at least one FXR agonist. In some embodiments, the at least one FXR agonist is identified by administering a test agent to a patient; determining the level of at least one of the following in the presence and/or absence of the test agent: (a) an extracellular matrix protein, (b) RAS pathway, (c) parathyroid hormone, (d) serum creatinine, (e) serum albumin, (f) proteinuria, (g) lipid metabolism, (h) renal lipid deposition, (i) mesangial expansion, (j) glomerulosclerosis, (k) kidney inflammation, (l) blood pressure, (m) bone resorption, (n) a MMP, and (o) bone formation; and selecting a FXR agonist which has at least one property selected from reducing the level of at least one of: (a) an extracellular matrix protein, (b) RAS pathway, (c) parathyroid hormone, (d) serum creatinine, (e) serum albumin, (f) proteinuria, (g) lipid metabolism, (h) renal lipid deposition, (i) mesangial expansion, (j) glomerulosclerosis, (k) kidney inflammation, (l) blood pressure, (m) bone resorption, and (n) a MMP, and elevating the level of bone formation in the patient.

Also provided are methods of diagnosing the risk that a patient will develop at least one condition that can be treated by elevating the VDR activity level. The methods include measuring the level of FXR activity in at least one cell of the patient.

Also provided are methods of characterizing the level of FXR activity in a mammal. The methods include determining the level of CYP27B1 in the mammal and characterizing the level of FXR activity in the mammal on the basis of the level of CYP27B1.

### DESCRIPTION OF DRAWINGS

FIG. 1 shows the level of CYP27B1 in wild and farnesoid X receptor deficient mice administered vehicle or FXR agonist, Compound A (isopropyl 3-(3,4-difluorobenzoyl)-1,1-dimethyl-1,2,3,6-tetrahydroazepino[4,5-b]indole-5-carboxylate) for 7 days.

FIG. 2 shows the serum creatinine level in KKAy, db/db, C57B1/6, FXR deficient, and LDLR deficient mice.

FIG. 3 shows the serum creatinine level in chow fed mice administered vehicle or Compound A for 7 days.

FIG. 4 shows the level of CYP27B1 in chow fed mice administered vehicle or Compound A for 7 days.

FIG. 5 shows the level of MMP-14 in chow fed mice administered vehicle or Compound A for 7 days.

FIG. 6 shows the level of renin in chow fed mice administered vehicle or Compound A for 28 days.

FIG. 7 shows an evaluation of the bone mass density in the distal femoral trabecular bone in female wild type and FXR −/− mice at 16 weeks and 22 weeks.

FIG. 8 shows an evaluation of the bone mass density in distal cortical femur in female wild type and FXR −/− mice at 16 weeks and 22 weeks.

FIG. 9A shows a graph of trabecular bone density at the distal femoral metaphysis in the femurs of male and female FXR −/− and wild type mice at 22, 28, 37, and 68 weeks.

FIG. 9B shows a graph of trabecular bone volume at the distal femoral metaphysis in the femurs of male and female FXR −/− and wild type mice at 22, 28, 37, and 68 weeks.

FIG. 10A shows a graph of cortical bone density at the femoral diaphysis of male and female FXR −/− and wild type mice at 22, 28, 37, and 68 weeks.

FIG. 10B shows a graph of cortical bone thickness at the femoral diaphysis of male and female FXR −/− and wild type mice at 22, 28, 37, and 68 weeks.

FIG. 11A shows images of distal femurs of female FXR −/− and wild type mice at age 22 weeks.

FIG. 11B shows images of distal femurs of male FXR −/− and wild type mice at age 22 weeks.

FIG. 12A compares the bone formation rate at the distal femoral metaphysis of female and male FXR −/− and wild type mice at 22 weeks.

FIG. 12B compares the mineral apposition rate at the distal femoral metaphysis of female and male FXR −/− and wild type mice at 22 weeks.

FIG. 13A compares the mineralized surface at the distal femoral metaphysis of female and male FXR −/− and wild type mice at 22 weeks.

FIG. 13B compares the eroded surface at the distal femoral metaphysis of female and male FXR −/− and wild type mice at 22 weeks.

FIG. 14 compares the bone turnover rate at the distal femoral metaphysis of female and male FXR −/− and wild type mice at 22 weeks.

FIG. 15 shows histological images of distal femurs of female and male FXR −/− and wild type mice. The images are magnified 200×. The thick arrows show double labeled surfaces and the thin arrows eroded surfaces.

FIG. 16A shows the level of serum calcium in FXR −/− and wild type mice at 12, 30, 41, and 51 weeks of age.

FIG. 16B shows the level of serum phosphate in FXR −/− and wild type mice at 12, 30, 41, and 51 weeks of age.

FIG. 17A shows reactions of vitamin D₃ catalyzed by the indicated enzymes to make the indicated derivatives.

FIG. 17B shows Cyp27a1 mRNA levels in FXR −/− and wild type mice at 12, 30, 41, and 51 weeks of age.

FIG. 17C shows Cyp27b1 mRNA levels in FXR −/− and wild type mice at 12, 30, 41, and 51 weeks of age.

FIG. 17D shows Cyp24a1 mRNA levels in FXR −/− and wild type mice at 12, 30, 41, and 51 weeks of age.

FIG. 18A shows body weight of female and male FXR −/− and wild type mice at 22 weeks of age.

FIG. 18B shows femoral length of female and male FXR −/− and wild type mice at 22 weeks of age.

FIG. 19 shows expression of FXR mRNA in mouse and human samples.

As used in the present specification, the following words and phrases are generally intended to have the meanings as set forth below, except to the extent that the context in which they are used indicates otherwise.

As used herein, “treating” refers to any manner in which at least one symptom or feature of a disease or disorder is beneficially altered so as to delay the onset, retard the progression, or ameliorate the symptoms of the disease or disorder. In some embodiments, an existing disease is treated. In some embodiments, a patient who has not yet manifested a symptom or feature of a disease or disorder is treated. In some embodiments, a patient who has not yet manifested a symptom or feature of a disease or disorder, but who has manifested at least one risk factor for development of the disease or disorder is treated. In some embodiments the at least one risk factor is a the presence of a genotypic marker of predisposition to development of the disease or disorder.

As used herein, “preventing” refers to administration of an agent to a patient so as to prevent the patient from developing a disease or disorder. In some embodiments prevention is measured over a finite period of time such as one month, three months, six months, one year, five years, ten years, or longer.

As used herein, the term “farnesoid X receptor (FXR)” refers to all mammalian forms of such receptor including, for example, alternative splice isoforms and naturally occurring isoforms (see, e.g. Huber et al, Gene (2002), Vol. 290, pp.: 35-43). Representative farnesoid X receptor species include, without limitation the rat (GenBank Accession No. NM_{—}021745), mouse (Genbank Accession No. NM_{—}009108), and human (GenBank Accession No. NM_{—}005123) forms of the receptor.

As used herein, “vitamin D receptor (VDR)” refers to all mammalian forms of such receptor including, for example, alternative splice isoforms and naturally occurring isoforms. Representative VDR species include, without limitation the rat (GenBank Accession No. NM_{—}01705), mouse (Genbank Accession No. NM_{—}009504), human variant 1 (GenBank Accession No. NM_{—}000376), and human variant 2 (GenBank Accession No. NM_{—}001017535) forms of the receptor.

As used herein, “Cytochrome P450, family 27, subfamily B, polypeptide 1 (CYP27B1)” refers to a gene that encodes for the protein 25-hydroxyvitamin D-1α-hydroxylase. As used herein, 25-hydroxyvitamin D-1α-hydroxylase refers to all mammalian forms of the protein, including for example, alternative splice isoforms and naturally occurring isoforms. Representative 25-hydroxyvitamin D-1α-hydroxylase species include, without limitation the mouse (GenBank Accession No. NM_{—}010009), rat (Genbank Accession No. NM_{—}053763), and human (GenBank Accession No. NM_{—}000785) forms.

As used herein, unless specified otherwise, a reference to “level” of a factor refers to the expression of a polynucleotide or gene encoding the factor or to the activity of the protein corresponding to the factor. Expression of a polynucleotide or gene can refer to the production of a RNA transcript (mRNA) or the production of a protein, so the level of a factor can be measured by assaying the amount of mRNA or protein produced. The level of a factor can also be measured by assaying the amount of activity of the protein produced.

In some embodiments, the level of CYP27B1 refers to expression of CYP27B1. In some embodiments, expression of CYP27B1 refers to the production of a RNA transcript (mRNA) of CYP27B1 or the production of the protein 25-hydroxyvitamin D-1α-hydroxylase which is encoded by CYP27B1. In some embodiments, the level of CYP27B1 is determined by measuring the level of CYP27B1 mRNA, level of 25-hydroxyvitamin D-1α-hydroxylase protein, or activity of 25-hydroxyvitamin D-1α-hydroxylase. 25-Hydroxyvitamin D₃ la-hydroxylase catalyzes the formation of 1α,25-dihydroxyvitamin D₃ from 25 hydroxyvitamin D₃. In some embodiments, the level of 25-hydroxyvitamin D-1α-hydroxylase is determined by measuring the amount of α,25-dihydroxyvitamin D₃. 1α,25 Dihydroxyvitamin D₃ refers to an active form of vitamin D that binds to VDRs and elevates the VDR activity level. In some embodiments, the level of CYP27B1 is determined by measuring the Vitamin D receptor activity level. In some embodiments, elevating the level of CYP27B1 elevates the level of 1α,25 dihydroxyvitamin D₃, to thereby elevate the level of VDR activity.

As used herein, “VDR activity” refers to at least one effect triggered by binding of a VDR ligand to VDR. In some embodiments, the VDR ligand is a VDR agonist. In some embodiments, the at least one effect triggered by binding of a VDR ligand includes transcriptional regulation of VDR target genes. In some embodiments, VDR activity increases transcription of VDR target genes. In some embodiments, VDR activity decreases transcription of VDR target genes. In some embodiments, transcription of renin is reduced by VDR activity. In some embodiments, a VDR ligand is 1α,25-dihydroxyvitamin D₃. In some embodiments, a VDR agonist is 1α,25-dihydroxyvitamin D₃.

As used herein, the term “agonist” refers to an agent that triggers a response that is at least one response or partial response triggered by binding of an endogenous ligand of the receptor to the receptor. In some embodiments, the agonist may act directly or indirectly on a second agent that itself modulates the activity of the receptor. In some embodiments, the at least one response of the receptor is an activity of the receptor that can be measured with assays including but not limited to physiological, pharmacological, and biochemical assays. Exemplary assays include but are not limited to assays that measure the binding of an agent to the receptor, the binding of the receptor to a substrate such as but not limited to a nuclear receptor and a regulatory element of a target gene, the effect on gene expression assayed at the mRNA or resultant protein level, and the effect on an activity of proteins regulated either directly or indirectly by the receptor.

An FXR agonist can elevate the level of CYP27B1 expression and elevate the level of the protein 25-hydroxyvitamin D₃ 1α-hydroxylase. Elevated levels of 25-hydroxyvitamin D₃ la-hydroxylase can elevate the level of 1α,25-dihydroxyvitamin D₃, to thereby elevate the VDR activity level. In some embodiments, FXR agonist activity is measured by monitoring the level of at least one of CYP27B1,25-hydroxyvitamin D₃ 1α-hydroxylase, 1α,25-dihydroxyvitamin D₃, and VDR activity. In some embodiments, FXR agonist administration to a patient elevates at least one of the level of CYP27B1, 25-hydroxyvitamin D₃ la-hydroxylase, 1α,25-dihydroxyvitamin D₃, and VDR activity.

As used herein, a “condition that can be treated by elevating the VDR activity level” in a patient refers to a disease or disorder in which elevating the VDR activity level can beneficially alter at least one symptom or feature of the disease or disorder so as to prevent or delay the onset, retard the progression, or ameliorate the symptoms of the disease or disorder. In some embodiments, treating a patient with at least one FXR agonist elevates the level of CYP27B1 in the patient. In some embodiments, the condition that can be treated by elevating the VDR activity level is characterized by deficient VDR activity levels in the patient. In some embodiments, the condition is at least one of obesity, glucose intolerance, diabetes, metabolic syndrome, chronic kidney disease, cardiovascular disease, and bone disease.

Chronic kidney disease occurs when a patient's kidneys partly or completely lose their ability to carry out normal functions gradually over time. Water, waste, and toxic substances build up in the body when kidney function is compromised. Conditions including but not limited to anemia, high blood pressure, acidosis, cholesterol and fatty acid disorders, cardiovascular disease, and bone disease can occur because of impaired kidney function. In some embodiments, the chronic kidney disease is characterized by at least one of diabetic nephropathy and renal failure. In some embodiments, the cardiovascular disease is characterized by at least one of coronary heart disease, cerebrovascular disease, peripheral vascular disease, congestive heart failure, myocardial infarction, left ventricular hypertrophy, hypertension, and atherosclerosis. In some embodiments, the bone disease is characterized by at least one of osteoporosis, osteomalacia, and rickets.

In some embodiments, treating a condition that can be treated by elevating the vitamin D receptor level in a patient with at least one FXR agonist reduces at least one feature of the condition. For example, in some embodiments treating chronic kidney disease with at least one FXR agonist reduces excess extracellular matrix production, lipid metabolism, renal lipid deposition, mesangial expansion, proteinuria, glomerulosclerosis, and kidney inflammation in the patient. In some embodiments, treatment with the at least one FXR agonist reduces the levels of albuminuria, a type of proteinuria, in the patient. In some embodiments, treating chronic kidney disease with a FXR agonist reduces the level of a matrix metalloprotease (MMP), an extracellular matrix protein, renin angiotensin system (RAS) pathway, parathyroid hormone, serum creatinine, and serum albumin in the patient. In some embodiments, treatment with at least one FXR agonist reduces the levels of renin, a component of the RAS pathway. In some embodiments, treatment with at least one FXR agonist reduces the level of at least one extracellular matrix protein, such as fibronectin and collagen IV. In some embodiments, elevated levels of at least one of serum creatinine, serum albumin, proteinuria, and albuminuria is indicative of kidney disease. In some embodiments, MMP activity modulates fibronectin and collagen IV levels. In some embodiments, FXR agonists reduce MMP levels, to thereby reduce levels of fibronectin and collagen IV. In some embodiments, MMPs reduce 1α,25 dihydroxyvitamin D₃ levels. In some embodiments, chronic kidney disease is associated with a secondary disorder in the patient including parathyroidism and cardiovascular disease. In some embodiments, treating chronic kidney disease with an FXR agonist treats the secondary disorder in the patient.

Treating cardiovascular disease with at least one FXR agonist can, for example, reduce the level of MMPs, parathyroid hormone, blood pressure, and RAS pathway in the patient. In some embodiments, treating bone disease reduces the level of parathyroid hormone in the patient, increases bone formation, and reduces bone resorption in the patient.

As used herein “parathyroid hormone” refers to all mammalian forms of such protein including, for example, alternative splice isoforms and naturally occurring isoforms. Representative renin species include, without limitation the mouse (GenBank Accession No. NM_{—}020623), rat (Genbank Accession No. NM_{—}017044), and human (GenBank Accession No. NM_{—}000315) forms of the protein.

Parathyroid hormone is secreted by the parathyroid gland and functions include without limitation regulation of Calcium homeostasis and bone resorption. In some embodiments, reducing parathyroid hormone levels reduces bone resorption.

As used herein “renin-angiotensin-system (RAS) pathway” refers to the level or activity of at least one component of the RAS pathway which regulates activities including but not limited to blood pressure, electrolyte balance, cardiac, and vascular functions in the body. Components of the RAS pathway include and are not limited to renin, angiotensinogen, Angiotensin I, Angiotensin II, and angiotensin converting enzyme (ACE). In some embodiments, reducing the level of the RAS pathway reduces the level of at least one of renin, Angiotensin I, and Angiotensin II. In the body, angiotensinogen is released from the liver and is converted to angiotensin I by the aspartyl protease renin. Angiotensin I is converted to angiotensin II by the carboxydipeptidase ACE. Angiotensin II acts as an endocrine hormone and an autocrine and paracrine effector in tissues including but not limited to kidney, heart, and brain. Angiotensin II activities include vasoconstriction; antinatriuretic, and dipsogenic activities and its activities can be mediated through angiotensin II receptors, type I and type II (AT₁ and AT₂). In some embodiments, treating with an FXR agonist reduces the level of renin to thereby reduce the level of at least one of Angiotensin I and Angiotensin II. In some embodiments, reducing the level of the RAS pathway treats at least one of chronic kidney disease and cardiovascular disease. In some embodiments, at least one of blood pressure and cardiovascular disease risk factors are reduced in patients treated with at least one FXR agonist.

As used herein, “renin” refers to all mammalian forms of such protein including, for example, alternative splice isoforms and naturally occurring isoforms. Representative renin species include, without limitation the mouse (GenBank Accession No. NM_{—}031192), rat (Genbank Accession No. NM_{—}012642), and human (GenBank Accession No. NM_{—}000537) forms of the protein.

As used herein, “MMP” refers to a member of the matrix metalloprotease family. There are at least twenty-five known members of the MMP family. In certain embodiments, a MMP is at least one of MMP-9 and MMP-14. As used herein, “matrix metalloproteinase-9 (MMP-9)” refers to all mammalian forms of the protein, including for example, alternative splice isoforms and naturally occurring isoforms. Representative MMP-9 species include, without limitation the human (GenBank Accession No. NM_{—}004994), mouse (Genbank Accession No. NM_{—}013599), and rat (GenBank Accession No. NM_{—}031055) forms. As used herein, “matrix metalloproteinase-14 (MMP-14)” refers to all mammalian forms of the protein, including for example, alternative splice isoforms and naturally occurring isoforms. Representative MMP-14 species include, without limitation, the human (GenBank Accession No. NM_{—}004995), mouse (GenBank Accession No. NM_{—}008608) and rat (GenBank Accession No. NM_{—}031056) forms.

As used herein the phrase “therapeutically effective amount” refers to the amount sufficient to provide a therapeutic outcome regarding at least one symptom or feature of a disease or condition.

As used herein, the phrase “effective amount” refers to the amount sufficient to increase or reduce a specified activity, function, or feature.

As used herein, “agent” refers to a substance including, but not limited to a chemical compound, such as a small molecule or a complex organic compound, a protein, such as an antibody or antibody fragment or a protein comprising an antibody fragment, or a genetic construct which acts at the DNA or mRNA level in an organism.

As used herein, reference to “modulate” refers to changing or altering an activity, function, or feature. The term “modulator” refers to an agent which modulates an activity, function, or feature. For example, an agent may modulate an activity by increasing or decreasing the activity compared to the effects on the activity in the absence of the agent. In some embodiments, a modulator can be an agonist.

Provided is a method of treating at least one condition that can be treated by elevating the vitamin D receptor (VDR) activity level in a patient by administering to the patient a therapeutically effective amount of at least one farnesoid X receptor (FXR) agonist. In some embodiments, the at least one FXR agonist elevates the level of Cytochrome P450, family 27, subfamily B, polypeptide 1 (CYP27B1), to thereby elevate the level of VDR activity in the patient. In some embodiments, the at least one condition is a disease characterized by deficient VDR activity levels in the patient. In some embodiments, the level of CYP27B1 is elevated in at least one cell type of the patient selected from kidney cells and bone cells. In some embodiments, the level of CYP27B1 is elevated in at least one bone cell type of the patient selected from osteoblasts and osteoclasts. In some embodiments, the at least one FXR agonist elevates the level of CYP27B1, to thereby elevate the level of 1α,25-dihydroxyvitamin D₃ in at least one of serum of the patient and a cell type of the patient selected from kidney cells and bone cells. In some embodiments, the level of 1α,25-dihydroxyvitamin D₃ is elevated in at least one bone cell type of the patient selected from osteoblasts and osteoclasts. In some embodiments, the VDR activity level is elevated in at least one cell type of the patient selected from kidney cells, cardiomyocytes, bone cells, immune cells, mesangial cells, and smooth muscle cells. In some embodiments, the VDR activity level is elevated in at least one bone cell type of the patient selected from osteoblasts and osteoclasts. In some embodiments, the VDR activity level is elevated in at least one immune cell type of the patient selected from dendritic cells, T lymphocytes, B lymphocytes, and monocytes. In some embodiments, administration of the at least one FXR agonist does not cause at least one of hypercalcemia and hypercalcinuria in the patient. In some embodiments, the at least one condition is selected from obesity, glucose intolerance, diabetes, and metabolic syndrome. In some embodiments, the at least one condition is chronic kidney disease. In some embodiments, the chronic kidney disease is characterized by at least one of diabetic nephropathy and renal failure. In some embodiments, treatment of the chronic kidney disease comprises treatment of at least one secondary disorder in the patient selected from parahyperthyroidism and cardiovascular disease. In some embodiments, the cardiovascular disease is characterized by at least one of coronary heart disease, cerebrovascular disease, peripheral vascular disease, congestive heart failure, myocardial infarction, left ventricular hypertrophy, hypertension, and atherosclerosis. In some embodiments, the at least one FXR agonist reduces the level of at least one of a matrix metalloprotease (MMP), an extracellular matrix protein, renin angiotensin system (RAS) pathway, parathyroid hormone, serum creatinine, serum albumin, proteinuria, lipid metabolism, renal lipid deposition, mesangial expansion, glomerulosclerosis, and kidney inflammation in the patient. In some embodiments, the at least one MMP is selected from MMP-9 and MMP-14. In some embodiments, the at least one extracellular matrix protein is selected from collagen IV and fibronectin. In some embodiments, the level of the RAS pathway is characterized by the level of renin in the patient. In some embodiments, the proteinuria is characterized by albuminuria in the patient. In some embodiments, the at least one condition is cardiovascular disease. In some embodiments, the cardiovascular disease is characterized by at least one of coronary heart disease, cerebrovascular disease, peripheral vascular disease, congestive heart failure, myocardial infarction, left ventricular hypertrophy, hypertension, and atherosclerosis. In some embodiments, the at least one FXR agonist reduces the level of at least one of a MMP, parathyroid hormone, blood pressure, and RAS pathway in the patient. In some embodiments, the at least one MMP is selected from MMP-9 and MMP-14. In some embodiments, the level of the RAS pathway is characterized by the level of renin in the patient. In some embodiments, the at least one condition is a bone disease. In some embodiments, the at least one bone disease is characterized by at least one of osteoporosis, osteomalacia, and rickets. In some embodiments, the at least one FXR agonist reduces the level of at least one of parathyroid hormone, bone turnover, and bone resorption in the patient. In some embodiments, the at least one FXR agonist elevates the level of bone formation in the patient.

Also provided is a method of modulating the level of at least one of CYP27B1 and 1α,25-dihydroxyvitamin D₃ in a cell by providing an effective amount of at least one FXR modulator, to thereby modulate the level of at least one of CYP27B1 and 1α,25-dihydroxyvitamin D₃ in the cell. In some embodiments, the level of at least one of CYP27B1 and 1α,25-dihydroxyvitamin D₃ is elevated in the cell and the at least one FXR modulator is a FXR agonist.

Also provided is a method of modulating the VDR activity level in a patient by administering to the patient an effective amount of at least one FXR modulator, to thereby modulate the VDR activity level in the patient. In some embodiments, the VDR activity level is elevated in the patient and the at least one FXR modulator is a FXR agonist.

Also provided is a method of modulating the level of at least one of an extracellular matrix protein, RAS pathway, parathyroid hormone, serum creatinine, serum albumin, proteinuria, lipid metabolism, renal lipid deposition, mesangial expansion, glomerulosclerosis, kidney inflammation, blood pressure, bone resorption, and bone formation in a patient by administering to the patient an effective amount of at least one FXR modulator. In some embodiments, the FXR modulator modulates the level of CYP27B1 in the patient, to thereby modulate the level of at least one of an extracellular matrix protein, RAS pathway, parathyroid hormone, serum creatinine, serum albumin, proteinuria, lipid metabolism, renal lipid deposition, mesangial expansion, glomerulosclerosis, kidney inflammation, blood pressure, bone resorption, and bone formation in the patient. In some embodiments, the at least one extracellular matrix protein is selected from collagen IV and fibronectin. In some embodiments, the level of the RAS pathway is characterized by the level of renin in the patient. In some embodiments, the proteinuria is characterized by albuminuria in the patient. In some embodiments, the level of CYP27B1 is elevated in the patient; the level of at least one of an extracellular matrix protein, renin angiotensin system (RAS) pathway, parathyroid hormone, serum creatinine, serum albumin, proteinuria, lipid metabolism, renal lipid deposition, mesangial expansion, glomerulosclerosis, and kidney inflammation, blood pressure, and bone resorption is reduced in the patient; and the at least one FXR modulator is a FXR agonist. In some embodiments, the level of CYP27B1 is elevated in the patient; the level of bone formation is elevated in the patient; and the at least one FXR modulator is a FXR agonist.

Also provided is a method of identifying a FXR modulator by providing a test agent to a cell; determining the level of at least one of CYP27B1 and 1α,25-dihydroxyvitamin D₃ in the presence of the test agent; comparing the level of the at least one of CYP27B1 and 1α,25-dihydroxyvitamin D₃ in the presence of the test agent to the level of the at least one of CYP27B1 and 1α,25-dihydroxyvitamin D₃ in the absence of the test agent; and identifying the test agent as a FXR modulator if the level of the at least one of CYP27B1 and 1α,25-dihydroxyvitamin D₃ is modulated in the presence of the test agent compared to the level of the at least one of CYP27B1 and 1α,25-dihydroxyvitamin D₃ in the absence of the test agent. In some embodiments the FXR modulator is a FXR agonist and the FXR agonist elevates the level of at least one of CYP27B1 and 1α,25-dihydroxyvitamin D₃ compared to the level of the at least one of CYP27B1 and 1α,25-dihydroxyvitamin D₃ in the absence of the test agent. Also provided is a method of treating at least one condition that can be treated by elevating the VDR activity level in a patient, by administering to the patient a therapeutically effective amount of at least one FXR agonist, wherein the at least one FXR agonist is identified by the method just described.

Also provided is a method of identifying a FXR modulator by administering a test agent to a patient; determining the VDR activity level in the patient; comparing the VDR activity level in the presence of the test agent to the VDR activity level in the absence of the test agent; and identifying the test agent as a FXR modulator if the VDR activity level is modulated in the presence of the test agent compared to its state in the absence of the test agent. In some embodiments the FXR modulator is a FXR agonist and wherein the FXR agonist elevates the VDR activity level in the patient relative to the VDR activity level in the absence of the test agent. Also provided is a method of treating at least one condition that can be treated by elevating the VDR activity level in a patient, the method comprising administering to the patient a therapeutically effective amount of at least one FXR agonist, wherein the at least one FXR agonist is identified by the method just described.

Also provided is a method of identifying a FXR modulator by administering a test agent to a patient; determining the level of at least one of the following factors in the presence the test agent: (a) an extracellular matrix protein, (b) RAS pathway, (c) parathyroid hormone, (d) serum creatinine, (e) serum albumin, (f) proteinuria, (g) lipid metabolism, (h) renal lipid deposition, (i) mesangial expansion, (j) glomerulosclerosis, (k) kidney inflammation, (l) blood pressure, (m) bone resorption, and (n) bone formation in the patient; comparing the level of at least one factor in the presence of the test agent to the level of the at least one factor in the absence of the test agent; and identifying the test agent as a FXR modulator if it modulates the level of at least one of: (a) an extracellular matrix protein, (b) RAS pathway, (c) parathyroid hormone, (d) serum creatinine, (e) serum albumin, (f) proteinuria, (g) lipid metabolism, (h) renal lipid deposition, (i) mesangial expansion, (j) glomerulosclerosis, (k) kidney inflammation, (l) blood pressure, (m) bone resorption, and (n) bone formation in the patient. In some embodiments the at least one extracellular matrix protein is selected from collagen IV and fibronectin. In some embodiments the level of the RAS pathway is characterized by the level of renin in the patient. In some embodiments the proteinuria is characterized by albuminuria in the patient. In some embodiments the FXR modulator is a FXR agonist and the FXR agonist has at least one property selected from reducing the level of at least one of: (a) an extracellular matrix protein, (b) renin angiotensin system (RAS) pathway, (c) parathyroid hormone, (d) serum creatinine, (e) serum albumin, (f) proteinuria, (g) lipid metabolism, (h) renal lipid deposition, (i) mesangial expansion, (j) glomerulosclerosis, (k) kidney inflammation, (l) blood pressure, and (m) bone resorption, and elevating the level of bone formation in the patient. Also provided is a method of treating at least one condition that can be treated by elevating the VDR activity level in a patient by administering to the patient a therapeutically effective amount of at least one FXR agonist, wherein the at least one FXR agonist is identified by the method just described. In some embodiments, the at least one extracellular matrix protein is selected from collagen IV and fibronectin. In some embodiments, the level of the RAS pathway is characterized by the level of renin in the patient. In some embodiments, the proteinuria is characterized by albuminuria in the patient. In some embodiments, the at least one MMP is selected from MMP-9 and MMP-14.

Also provided is a method of diagnosing the risk that a patient will develop at least one condition that can be treated by elevating the VDR activity level in the patient, by measuring the level of FXR activity in at least one cell of the patient; comparing the level of FXR activity in the patient to a reference standard; and diagnosing an increased risk that the patient will develop at least one condition that can be treated by elevating the VDR activity level in the patient if the level of FXR activity in the patient is below the reference standard.

Also provided is method of characterizing the level of FXR activity in a mammal by determining the level of CYP27B1 in the mammal and characterizing the level of FXR activity in the mammal on the basis of the level of CYP27B1. In some embodiments, the level of CYP27B1 is above about a predetermined threshold and the level of FXR activity is determined to be above about a predetermined threshold. In some embodiments, the level of CYP27B1 is below about a predetermined threshold and the level of FXR activity is determined to be below about a predetermined threshold. In some embodiments, the level of FXR activity in the mammal is determined to be characteristic of a disease state. In some embodiments, the mammal is a human.

In some embodiments provided herein the FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl) 1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoy1)-1,4,4-trimethy1-1,4,5,6-tetrahydro-pyrro1o[2,3-d]azepine-2,8-dicarboxylic acid 2-ethy1 ester 8-isopropy1 ester;
6-(3,4-difluoro-benzoy1)-4,4-dimethy1-1,4,5,6-tetrahydro-pyrro1o[2,3-d]azepine-2,8-dicarboxylic acid 2-ethy1 ester 8-isopropy1 ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro-4H-thieno[2,3-D]azepine-8-carboxylic acid ethyl ester;
6-(4-fluoro-benzoyl)-3,6,7,8-tetrahydro-imidazo[4,5-D]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3(4-fluorobenzoyl)-2-methyl-1,2,3,6-tetrahydroazepino[4,5-b]indole-5-carboxylate; and
n-propyl 3(4-fluorobenzoyl)-2-methyl-8-fluoro-1,2,3,6-tetrahydroazepino[4,5-b]indole-5-carboxylate.

In some embodiments of the methods the FXR agonist or modulator is selected from a compound disclosed in at least one of U.S. Patent Application Publication No. 2004/0023947A1, published Feb. 5, 2004, U.S. Patent Application Publication No. 2005/0054634A1, published Mar. 10, 2005, U.S. Patent Application Publication No. 2007/0015746A1, published Jan. 18, 2007, and International Patent Application Publication No. 2007/070796, published Jun. 21, 2007, each of which are hereby incorporated herein by reference in their entirety.

Pharmaceutical compositions for use in the methods herein are formulated to contain therapeutically effective amounts of at least one FXR modulator or pharmaceutically acceptable derivative. The pharmaceutical compositions are useful, for example, in the treatment of at least one condition that can be treated by elevating the vitamin D receptor (VDR) activity level in a patient. In some embodiments, the pharmaceutical compositions are useful in the treatment of at least one disease characterized by deficient VDR activity levels in the patient. In some embodiments, the pharmaceutical compositions are useful in the treatment of at least one condition selected from obesity, glucose intolerance, diabetes, metabolic syndrome, chronic kidney disease, cardiovascular disease, and bone disease.

Pharmaceutically acceptable derivatives include acids, bases, enol ethers and esters, salts, esters, hydrates, solvates and prodrug forms. The derivative is selected such that its pharmacokinetic properties are superior with respect to at least one characteristic to the corresponding neutral agent. The FXR modulator may be derivatized prior to formulation.

In some embodiments, the at least one FXR modulator or pharmaceutically acceptable derivative is formulated into a suitable pharmaceutical preparation such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the FXR modulator or pharmaceutically acceptable derivative is formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more FXR modulators or pharmaceutically acceptable derivatives are mixed with a suitable pharmaceutical carrier or vehicle.

The concentrations of the FXR modulator or pharmaceutically acceptable derivative in the compositions are effective for delivery of an amount, upon administration, that treats one or more of the symptoms of at least one condition that can be treated by elevating the VDR activity level in a patient. In some embodiments, the least one condition is a disease characterized by deficient VDR activity levels in the patient. In some embodiments, the at least one condition is selected from obesity, glucose intolerance, diabetes, metabolic syndrome, chronic kidney disease, cardiovascular disease, and bone disease.

Typically, by way of example and without limitation, the compositions are formulated for single dosage administration. To formulate a composition, the weight fraction of the FXR modulator or pharmaceutically acceptable derivative is dissolved, suspended, dispersed or otherwise mixed in a selected vehicle at an effective concentration such that the treated condition is relieved or ameliorated. Pharmaceutical carriers or vehicles suitable for administration of the FXR modulator or pharmaceutically acceptable derivative include any such carriers known to those skilled in the art to be suitable for the particular mode of administration.

In addition, the FXR modulator or pharmaceutically acceptable derivative can be formulated as the sole modulator in the composition or can be combined with other modulators. Liposomal suspensions, including tissue-targeted liposomes, can also be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) can be formed by drying down egg phosphatidyl choline and brain phosphatidyl serine (7:3 molar ratio) on the inside of a flask. A solution of a FXR modulator or pharmaceutically acceptable derivative provided herein in phosphate buffered saline lacking divalent cations (PBS) is added and the flask shaken until the lipid film is dispersed. The resulting vesicles are washed to remove unencapsulated FXR modulator or pharmaceutically acceptable derivative, pelleted by centrifugation, and then resuspended in PBS.

The FXR modulator or pharmaceutically acceptable derivative is included in the pharmaceutically acceptable carrier in an amount sufficient to exert a therapeutically useful effect in the absence of undesirable side effects on the patient treated. The therapeutically effective concentration may be determined empirically by testing the compositions in in vitro and in vivo systems described herein and in International Patent Application Publication Nos. 99/27365 and 00/25134 and then extrapolated there from for dosages for humans.

The concentration of the FXR modulator or pharmaceutically acceptable derivative in the pharmaceutical composition will depend on absorption, inactivation, and excretion rates of the modulator, the physicochemical characteristics of the modulator, the dosage schedule, and amount administered as well as other factors known to those of skill in the art. For example, the amount that is delivered is sufficient to treat at least one condition that can be treated by elevating the VDR activity level in a patient. In some embodiments, the amount that is delivered is sufficient to treat at least one disease characterized by deficient VDR activity levels in the patient. In some embodiments, the amount that is delivered is sufficient to treat at least one condition selected from obesity, glucose intolerance, diabetes, chronic kidney disease, cardiovascular disease, and bone disease.

Typically a therapeutically effective dosage should produce a serum concentration of FXR modulator or pharmaceutically acceptable derivative of from about 0.1 ng/ml to about 50-100 μg/ml. The pharmaceutical compositions typically should provide a dosage of from about 0.001 mg to about 2000 mg of FXR modulator or pharmaceutically acceptable derivative per kilogram of body weight per day. Pharmaceutical dosage unit forms are prepared to provide from about 1 mg to about 1000 mg, such as from about 10 to about 500 mg of the FXR modulator or pharmaceutically acceptable derivative or a combination of modulators per dosage unit form.

The FXR modulator or pharmaceutically acceptable derivative may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed methods.

Thus, effective concentrations or amounts of one or more FXR modulators or pharmaceutically acceptable derivatives thereof are mixed with a suitable pharmaceutical carrier or vehicle for systemic, topical, or local administration to form pharmaceutical compositions. FXR modulators or pharmaceutically acceptable derivatives are included in an amount effective for treating at least one condition that can be treated by elevating the VDR activity level in a patient. In some embodiments, FXR modulators or pharmaceutically acceptable derivatives are included in an amount effective for treating at least one disease characterized by deficient VDR activity levels in the patient. In some embodiments, FXR modulators or pharmaceutically acceptable derivatives are included in an amount effective for treating at least one condition selected from obesity, glucose intolerance, diabetes, metabolic syndrome, chronic kidney disease, cardiovascular disease, and bone disease. The concentration of FXR modulator or pharmaceutically acceptable derivative in the composition will depend on absorption, inactivation, excretion rates of the FXR modulator or pharmaceutically acceptable derivative, the dosage schedule, amount administered, particular formulation as well as other factors known to those of skill in the art.

The compositions are intended to be administered by a suitable route, including by way of example and without limitation orally, parenterally, rectally, topically and locally. For oral administration, capsules and tablets can be used. The compositions are in liquid, semi-liquid or solid form and are formulated in a manner suitable for each route of administration.

Solutions or suspensions used for parenteral, intradermal, subcutaneous, or topical application can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampoules, disposable syringes or single or multiple dose vials made of glass, plastic or other suitable material.

In instances in which the FXR modulators or pharmaceutically acceptable derivatives exhibit insufficient solubility, methods for solubilizing the FXR modulators or pharmaceutically acceptable derivatives may be used. Such methods are known to those of skill in this art, and include, but are not limited to, using co-solvents, such as dimethylsulfoxide (DMSO), using surfactants, such as TWEEN®, or dissolution in aqueous sodium bicarbonate. Pharmaceutically acceptable derivatives of the FXR modulators may be used in formulating effective pharmaceutical compositions.

Upon mixing or addition of the FXR modulator or pharmaceutically acceptable derivative(s), the resulting mixture may be a solution, suspension, emulsion or the like. The form of the resulting mixture depends upon a number of factors, including the intended mode of administration and the solubility of the FXR modulator or pharmaceutically acceptable derivative in the selected carrier or vehicle. The effective concentration is sufficient for treating one or more symptoms of at least one condition that can be treated by elevating the VDR activity level in a patient and may be empirically determined. In some embodiments, the effective concentration is sufficient for treating one or more symptoms of at least one disease characterized by deficient VDR activity levels in the patient. In some embodiments, the effective concentration is sufficient for treating one or more symptoms of at least one condition selected from obesity, glucose intolerance, diabetes, metabolic syndrome, chronic kidney disease, cardiovascular disease, and bone disease.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the agents or pharmaceutically acceptable derivatives thereof. The FXR modulator or pharmaceutically acceptable derivative thereof is typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the FXR modulator or pharmaceutically acceptable derivative sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the FXR modulator or pharmaceutically acceptable derivative, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an modulator as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without-limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art; for example, see Remington: The Science and Practice of Pharmacy. 21^{st} Edition. Philadelphia, Pa. Lippincott Williams & Wilkins. 2005. The composition or formulation to be administered will, in any event, contain a quantity of the FXR modulator or pharmaceutically acceptable derivative in an amount sufficient to alleviate the symptoms of the treated subject.

Dosage forms or compositions containing FXR modulator or pharmaceutically acceptable derivative in the range of 0.005% to 100% with the balance made up from non-toxic carrier may be prepared. For oral administration, a pharmaceutically acceptable non-toxic composition is formed by the incorporation of any of the normally employed excipients, such as, for example and without limitation, pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% FXR modulator or pharmaceutically acceptable derivative, such as 0.1-85%, or such as 75-95%.

The FXR modulator or pharmaceutically acceptable derivative may be prepared with carriers that protect the modulator or pharmaceutically acceptable derivative against rapid elimination from the body, such as time release formulations or coatings. The compositions may include other modulators to obtain desired combinations of properties. FXR modulators or pharmaceutically acceptable derivatives thereof, may also be advantageously administered for therapeutic or prophylactic purposes together with another pharmacological agent or modulator known in the general art to be of value in treating at least one condition that can be treated by elevating the VDR activity level in a patient, in treating at least one disease characterized by deficient VDR activity levels in the patient, or in treating at least one condition selected from obesity, glucose intolerance, diabetes, metabolic syndrome, chronic kidney disease, cardiovascular disease, and bone disease.

Oral pharmaceutical dosage forms include, by way of example and without limitation, solid, gel and liquid. Solid dosage forms include tablets, capsules, granules, and bulk powders. Oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In some embodiments, the formulations are solid dosage forms, such as capsules or tablets. The tablets, pills, capsules, troches and the like can contain any of the following ingredients, or agents of a similar nature: a binder; a diluent; a disintegrating agent; a lubricant; a glidant; a sweetening agent; and a flavoring agent.

Examples of binders include, by way of example and without limitation, microcrystalline cellulose, gum tragacanth, glucose solution, acacia mucilage, gelatin solution, sucrose, and starch paste. Lubricants include, by way of example and without limitation, talc, starch, magnesium or calcium stearate, lycopodium and stearic acid. Diluents include, by way of example and without limitation, lactose, sucrose, starch, kaolin, salt, mannitol, and dicalcium phosphate. Glidants include, by way of example and without limitation, colloidal silicon dioxide. Disintegrating agents include, by way of example and without limitation, crosscarmellose sodium, sodium starch glycolate, alginic acid, corn starch, potato starch, bentonite, methylcellulose, agar and carboxymethylcellulose. Coloring agents include, by way of example and without limitation, any of the approved certified water soluble FD and C dyes, mixtures thereof; and water insoluble FD and C dyes suspended on alumina hydrate. Sweetening agents include, by way of example and without limitation, sucrose, lactose, mannitol and artificial sweetening agents such as saccharin, and any number of spray dried flavors. Flavoring agents include, by way of example and without limitation, natural flavors extracted from plants such as fruits and synthetic blends of agents which produce a pleasant sensation, such as, but not limited to peppermint and methyl salicylate. Wetting agents include, by way of example and without limitation, propylene glycol monostearate, sorbitan monooleate, diethylene glycol monolaurate, and polyoxyethylene laural ether. Emetic-coatings include, by way of example and without limitation, fatty acids, fats, waxes, shellac, ammoniated shellac and cellulose acetate phthalates. Film coatings include, by way of example and without limitation, hydroxyethylcellulose, sodium carboxymethylcellulose, polyethylene glycol 4000 and cellulose acetate phthalate.

If oral administration is desired, the FXR modulator or pharmaceutically acceptable derivative could be provided in a composition that protects it from the acidic environment of the stomach. For example, the composition can be formulated in an enteric coating that maintains its integrity in the stomach and releases the modulator in the intestine. The composition may also be formulated in combination with an antacid or other such ingredient.

When the dosage unit form is a capsule, it can contain, in addition to material of the above, a liquid carrier such as a fatty oil. In addition, dosage unit forms can contain various other materials which modify the physical form of the dosage unit, for example, coatings of sugar and other enteric agents. The FXR modulator or pharmaceutically acceptable derivative can also be administered as a component of an elixir, suspension, syrup, wafer, sprinkle, chewing gum or the like. A syrup may contain, in addition to the modulators, sucrose as a sweetening agent and certain preservatives, dyes and colorings and flavors.

The FXR modulator or pharmaceutically acceptable derivative can also be mixed with other agents which do not impair the desired action, or with materials that supplement the desired action, such as antacids, H2 blockers, and diuretics.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents may be used in any of the above dosage forms.

Solvents, include by way of example and without limitation, glycerin, sorbitol, ethyl alcohol and syrup. Examples of preservatives include without limitation glycerin, methyl and propylparaben, benzoic add, sodium benzoate and alcohol. Non-aqueous liquids utilized in emulsions, include by way of example and without limitation, mineral oil and cottonseed oil. Emulsifying agents, include by way of example and without limitation, gelatin, acacia, tragacanth, bentonite, and surfactants such as polyoxyethylene sorbitan monooleate. Suspending agents include, by way of example and without limitation, sodium carboxymethylcellulose, pectin, tragacanth, Veegum and acacia. Diluents include, by way of example and without limitation, lactose and sucrose. Sweetening agents include, by way of example and without limitation, sucrose, syrups, glycerin and artificial sweetening agents such as saccharin. Wetting agents, include by way of example and without limitation, propylene glycol monostearate, sorbitan monooleate, diethylene glycol monolaurate, and polyoxyethylene lauryl ether. Organic acids include, by way of example and without limitation, citric and tartaric acid. Sources of carbon dioxide include, by way of example and without limitation, sodium bicarbonate and sodium carbonate. Coloring agents include, by way of example and without limitation, any of the approved certified water soluble FD and C dyes, and mixtures thereof. Flavoring agents include, by way of example and without limitation, natural flavors extracted from plants such fruits, and synthetic blends of agents which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the modulator or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a agent provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

Tablets and capsules formulations may be coated as known by those of skill in the art in order to modify or sustain dissolution of the FXR modulator or pharmaceutically acceptable derivative. Thus, for example and without limitation, they may be coated with a conventional enterically digestible coating, such as phenylsalicylate, waxes and cellulose acetate phthalate.

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients, include by way of example and without limitation, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a FXR modulator or pharmaceutically acceptable derivative is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The FXR modulator or pharmaceutically acceptable derivative diffuses through the outer polymeric membrane in a release rate controlling step. The percentage of FXR modulator or pharmaceutically acceptable derivative contained in such parenteral compositions is highly dependent on the specific nature thereof, as well as the activity of the FXR modulator or pharmaceutically acceptable derivative and the needs of the subject.

Parenteral administration of the FXR modulators or pharmaceutically acceptable derivatives includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Aqueous vehicles include, by way of example and without limitation, Sodium Chloride Injection, Ringers Injection, Isotonic Dextrose Injection, Sterile Water Injection, Dextrose and Lactated Ringers Injection. Nonaqueous parenteral vehicles include, by way of example and without limitation, fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include, by way of example and without limitation, sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include, by way of example and without limitation, ethyl alcohol, polyethylene glycol and propylene glycol for water miscible vehicles and sodium hydroxide, hydrochloric acid, citric acid or lactic acid for pH adjustment.

The concentration of the FXR modulator or pharmaceutically acceptable derivative is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. Preparations for parenteral administration should be sterile, as is known and practiced in the art.

Illustratively, intravenous or intraarterial infusion of a sterile aqueous solution containing a FXR modulator or pharmaceutically acceptable derivative is an effective mode of administration. Another embodiment is a sterile aqueous or oily solution or suspension containing an FXR modulator or pharmaceutically acceptable derivative injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the FXR modulator or pharmaceutically acceptable derivative to the treated tissue(s). The FXR modulator or pharmaceutically acceptable derivative may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The FXR modulator or pharmaceutically acceptable derivative may be suspended in micronized or other suitable form or may be derivatized, e.g., to produce a more soluble active product or to produce a prodrug or other pharmaceutically acceptable derivative. The form of the resulting mixture depends upon a number of factors, including the intended mode of administration and the solubility of the agent in the selected carrier or vehicle. The effective concentration is sufficient for ameliorating the symptoms of the condition and may be empirically determined.

Lyophilized powders can be reconstituted for administration as solutions, emulsions, and other mixtures or formulated as solids or gels.

The sterile, lyophilized powder is prepared by dissolving a FXR modulator or pharmaceutically acceptable derivative provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain, by way of example and without limitation, a single dosage (10-1000 mg, such as 100-500 mg) or multiple dosages of the agent. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, such as about 5-35 mg, for example, about 9-30 mg of lyophilized powder, is added per mL of sterile water or other suitable carrier. The precise amount depends upon the selected agent. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The FXR modulators or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, by way of example and without limitation, have diameters of less than about 50 microns, such as less than about 10 microns.

The FXR modulators or pharmaceutically acceptable derivatives may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the FXR modulator or pharmaceutically acceptable derivative alone or in combination with other pharmaceutically acceptable excipients can also be administered.

These solutions, particularly those intended for ophthalmic use, may be formulated, by way of example and without limitation, as about 0.01% to about 10% isotonic solutions, pH about 5-7, with appropriate salts.

Other routes of administration, such as transdermal patches, and rectal administration are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

Pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is, by way of example and without limitation, about 2 to 3 gm.

Tablets and capsules for rectal administration are manufactured using the same pharmaceutically acceptable substance and by the same methods as for formulations for oral administration.

The FXR modulators or pharmaceutically acceptable derivatives thereof, may also be formulated to be targeted to a particular tissue, receptor, or other area of the body of the subject to be treated. Many such targeting methods are well known to those of skill in the art. Such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a agent provided herein in phosphate buffered saline lacking divalent cations (PBS) is added and the flask shaken until the lipid film is dispersed. The resulting vesicles are washed to remove unencapsulated agent, pelleted by centrifugation, and then resuspended in PBS.

The FXR modulators or pharmaceutically acceptable derivatives for use in the methods may be packaged as articles of manufacture containing packaging material, a FXR modulator or pharmaceutically acceptable derivative thereof provided herein, which is effective for modulating the activity of a farnesoid X receptor or for treatment of one or more symptoms of at least one condition that can be treated by elevating the VDR activity level in a patient, within the packaging material, and a label that indicates that the FXR modulator or composition, or pharmaceutically acceptable derivative thereof, is used for modulating the activity of farnesoid X receptor for treatment of one or more symptoms of at least one condition that can be treated by elevating the VDR activity level in a patient. In some embodiments, the at least one condition is a disease characterized by deficient VDR activity levels in the patient. In some embodiments, the at least one condition is selected from obesity, glucose intolerance, diabetes, metabolic syndrome, chronic kidney disease, cardiovascular disease, and bone disease.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

Standard physiological, pharmacological and biochemical procedures are available for testing agents to identify those that possess biological activities that modulate the activity of the farnesoid X receptor. Such assays include, for example, biochemical assays such as binding assays, fluorescence polarization assays, FRET based coactivator recruitment assays (see generally Glickman et al., J. Biomolecular Screening, 7 No. 1 3-10 (2002)), as well as cell based assays including the co-transfection assay, the use of LBD-Gal 4 chimeras, protein-protein interaction assays (see, Lehmann. et al., J. Biol. Chem., 272(6) 3137-3140 (1997), and gene expression assays.

High throughput screening systems are commercially available (see, e.g., Zymark Corp., Hopkinton, Mass.; Air Technical Industries, Mentor, Ohio; Beckman Instruments Inc., Fullerton, Calif.; Precision Systems, Inc., Natick, Mass.) that enable these assays to be run in a high throughput mode. These systems typically automate entire procedures, including sample and reagent pipetting, liquid dispensing timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

Assays that do not require washing or liquid separation steps can be used for high throughput screening systems and include biochemical assays such as fluorescence polarization assays (see for example, Owicki, J., Biomol Screen 2000 October; 5(5):297) scintillation proximity assays (SPA) (see for example, Carpenter et al., Methods Mol Biol 2002; 190:31-49) and fluorescence resonance energy transfer energy transfer (FRET) or time resolved FRET based coactivator recruitment assays (Mukherjee et al., J Steroid Biochem Mol Biol 2002 July; 81(3):217-25; (Zhou et al., Mol. Endocrinol. 1998 October; 12(10):1594-604). Generally such assays can be preformed using either the full length receptor, or isolated ligand binding domain (LBD). In the case of the farnesoid X receptor, the LBD comprises amino acids 244 to 472 of the full length sequence.

If a fluorescently labeled ligand is available, fluorescence polarization assays provide a way of detecting binding of agents to the farnesoid X receptor by measuring changes in fluorescence polarization that occur as a result of the displacement of a trace amount of the label ligand by the agent. Additionally this approach can also be used to monitor the ligand dependent association of a fluorescently labeled coactivator peptide to the farnesoid X receptor to detect ligand binding to the farnesoid X receptor.

The ability of an agent to bind to a receptor, or heterodimer complex with RXR, can also be measured in a homogeneous assay format by assessing the degree to which the agent can compete off a radiolabelled ligand with known affinity for the receptor using a scintillation proximity assay (SPA). In this approach, the radioactivity emitted by a radiolabelled agent generates an optical signal when it is brought into close proximity to a scintillant such as a Ysi-copper containing bead, to which the farnesoid X receptor is bound. If the radiolabelled agent is displaced from the farnesoid X receptor the amount of light emitted from the farnesoid X receptor bound scintillant decreases, and this can be readily detected using standard microplate liquid scintillation plate readers such as, for example, a Wallac MicroBeta reader.

The heterodimerization of the farnesoid X receptor with RXRα can also be measured by fluorescence resonance energy transfer (FRET), or time resolved FRET, to monitor the ability of the agents provided herein to bind to the farnesoid X receptor or other nuclear receptors. Both approaches rely upon the fact that energy transfer from a donor molecule to an acceptor molecule only occurs when donor and acceptor are in close proximity. Typically the purified LBD of the farnesoid X receptor is labeled with biotin then mixed with stoichiometric amounts of europium labeled streptavidin (Wallac Inc.), and the purified LBD of RXRα is labeled with a suitable fluorophore such as CY5™. Equimolar amounts of each modified LBD are mixed together and allowed to equilibrate for at least 1 hour prior to addition to either variable or constant concentrations of the sample for which the affinity is to be determined. After equilibration, the time-resolved fluorescent signal is quantitated using a fluorescent plate reader. The affinity of the agent can then be estimated from a plot of fluorescence versus concentration of agent added.

This approach can also be exploited to measure the ligand dependent interaction of a co-activator peptide with a farnesoid X receptor in order to characterize the agonist or antagonist activity of the agents disclosed herein. Typically the assay in this case involves the use a recombinant Glutathione-S-transferase (GST)-farnesoid X receptor ligand binding domain (LBD) fusion protein and a synthetic biotinylated peptide sequenced derived from the receptor interacting domain of a co-activator peptide such as the steroid receptor coactivator 1 (SRC-1). Typically GST-LBD is labeled with a europium chelate (donor) via a europium-tagged anti-GST antibody, and the coactivator peptide is labeled with allophycocyanin via a streptavidin-biotin linkage.

In the presence of an agonist for the farnesoid X receptor, the peptide is recruited to the GST-LBD bringing europium and allophycocyanin into close proximity to enable energy transfer from the europium chelate to the allophycocyanin. Upon excitation of the complex with light at 340 nm excitation energy absorbed by the europium chelate is transmitted to the allophycocyanin moiety resulting in emission at 665 nm. If the europium chelate is not brought into close proximity to the allophycocyanin moiety there is little or no energy transfer and excitation of the europium chelate results in emission at 615 nm. Thus the intensity of light emitted at 665 nm gives an indication of the strength of the protein-protein interaction. The activity of a farnesoid X receptor antagonist can be measured by determining the ability of a agent to competitively inhibit (i.e., IC₅₀) the activity of an agonist for the farnesoid X receptor.

DNA binding assays can be used to evaluate the ability of an agent to modulate farnesoid X receptor activity. These assays measure the ability of nuclear receptor proteins, including farnesoid X receptor and RXR, to bind to regulatory elements of genes known to be modulated by farnesoid X receptor. In general, the assay involves combining a DNA sequence which can interact with the farnesoid X receptors, and the farnesoid X receptor proteins under conditions, such that the amount of binding of the farnesoid X receptor proteins in the presence or absence of the agent can be measured. In the presence of an agonist, farnesoid X receptor heterodimerizes with RXR and the complex binds to the regulatory element. Methods including, but not limited to DNAse footprinting, gel shift assays, and chromatin immunoprecipitation can be used to measure the amount of farnesoid X receptor proteins bound to regulatory elements.

In addition a variety of cell based assay methodologies may be successfully used in screening assays to identify and profile the specificity of agents described herein. These approaches include the co-transfection assay, translocation assays, and gene expression assays.

Three basic variants of the co-transfection assay strategy exist, co-transfection assays using full-length farnesoid X receptor, co-transfection assays using chimeric farnesoid X receptors comprising the ligand binding domain of the farnesoid X receptor fused to a heterologous DNA binding domain, and assays based around the use of the mammalian two hybrid assay system.

The basic co-transfection assay is based on the co-transfection into the cell of an expression plasmid to express the farnesoid X receptor in the cell with a reporter plasmid comprising a reporter gene whose expression is under the control of DNA sequence that is capable of interacting with that nuclear receptor. (See for example U.S. Pat. Nos. 5,071,773; 5,298,429, 6,416,957, WO 00/76523). Treatment of the transfected cells with an agonist for the farnesoid X receptor increases the transcriptional activity of that receptor which is reflected by an increase in expression of the reporter gene, which may be measured by a variety of standard procedures.

For those receptors that function as heterodimers with RXR, such as the farnesoid X receptor, the co-transfection assay typically includes the use of expression plasmids for both the farnesoid X receptor and RXR. Typical co-transfection assays require access to the full-length farnesoid X receptor and suitable response elements that provide sufficient screening sensitivity and specificity to the farnesoid X receptor.

Genes encoding the following full-length previously described proteins, which are suitable for use in the co-transfection studies and profiling the agents described herein, include rat farnesoid X receptor (GenBank Accession No. NM_{—}021745), human farnesoid X receptor (GenBank Accession No. NM_{—}005123), human RXR α (GenBank Accession No. NM_{—}002957), human RXR β (GenBank Accession No. XM_{—}042579), human RXR γ (GenBank Accession No. XM_{—}053680),

Reporter plasmids may be constructed using standard molecular biological techniques by placing cDNA encoding for the reporter gene downstream from a suitable minimal promoter. For example luciferase reporter plasmids may be constructed by placing cDNA encoding firefly luciferase immediately down stream from the herpes virus thymidine kinase promoter (located at nucleotides residues −105 to +51 of the thymidine kinase nucleotide sequence) which is linked in turn to the various response elements.

Numerous methods of co-transfecting the expression and reporter plasmids are known to those of skill in the art and may be used for the co-transfection assay to introduce the plasmids into a suitable cell. Typically such a cell will not endogenously express farnesoid X receptors that interact with the response elements used in the reporter plasmid.

Numerous reporter gene systems are known in the art and include, for example, alkaline phosphatase (See Berger, J., et al (1988) Gene 66 1-10; Kain, S. R. (1997) Methods. Mol. Biol. 63 49-60), β-galactosidase (See, U.S. Pat. No. 5,070,012, issued Dec, 3, 1991 to Nolan et al., and Bronstein, I., et al., (1989) J. Chemilum. Biolum. 4 99-111), chloramphenicol acetyltransferase (See Gorman et al., Mol Cell Biol. (1982) 2 1044-51), β-glucuronidase, peroxidase, β-lactamase (See U.S. Pat. Nos. 5,741,657 and 5,955,604), catalytic antibodies, luciferases (See U.S. Pat. Nos. 5,221,623; 5,683,888; 5,674,713; 5,650,289; 5,843,746) and naturally fluorescent proteins (See Tsien, R. Y. (1998) Annu. Rev. Biochem. 67 509-44).

The use of chimeras comprising the ligand binding domain (LBD) of the farnesoid X receptor fused to a heterologous DNA binding domain (DBD) expands the versatility of cell based assays by directing activation of the farnesoid X receptor in question to defined DNA binding elements recognized by defined DNA binding domain (see WO95/18380). This assay expands the utility of cell based co-transfection assays in cases where the biological response or screening window using the native DNA binding domain is not satisfactory.

In general the methodology is similar to that used with the basic co-transfection assay, except that a chimeric construct is used in place of the full-length farnesoid X receptor. As with the full-length farnesoid X receptor, treatment of the transfected cells with an agonist for the farnesoid X receptor LBD increases the transcriptional activity of the heterologous DNA binding domain which is reflected by an increase in expression of the reporter gene as described above. Typically for such chimeric constructs, the DNA binding domains from defined farnesoid X receptors, or from yeast or bacterially derived transcriptional regulators such as members of the GAL 4 and Lex A/Umud super families are used.

A third cell based assay of utility for screening agents is a mammalian two-hybrid assay that measures the ability of the nuclear hormone receptor to interact with a cofactor in the presence of a ligand. (See for example, U.S. Pat. Nos. 5,667,973, 5,283,173 and 5,468,614). The basic approach is to create three plasmid constructs that enable the interaction of the farnesoid X receptor with the interacting protein to be coupled to a transcriptional readout within a living cell. The first construct is an expression plasmid for expressing a fusion protein comprising the interacting protein, or a portion of that protein containing the interacting domain, fused to a GAL4 DNA binding domain. The second expression plasmid comprises DNA encoding the farnesoid X receptor fused to a strong transcription activation domain such as VP16, and the third construct comprises the reporter plasmid comprising a reporter gene with a minimal promoter and GAL4 upstream activating sequences.

Once all three plasmids are introduced into a cell, the GAL4 DNA binding domain encoded in the first construct allows for specific binding of the fusion protein to GAL4 sites upstream of a minimal promoter. However because the GAL4 DNA binding domain typically has no strong transcriptional activation properties in isolation, expression of the reporter gene occurs only at a low level. In the presence of a ligand, the farnesoid X receptor-VP16 fusion protein can bind to the GAL4-interacting protein fusion protein bringing the strong transcriptional activator VP16 in close proximity to the GAL4 binding sites and minimal promoter region of the reporter gene. This interaction significantly enhances the transcription of the reporter gene, which can be measured for various reporter genes as described above. Transcription of the reporter gene is thus driven by the interaction of the interacting protein and farnesoid X receptor in a ligand dependent fashion.

An agent can be tested for the ability to induce nuclear localization of a nuclear protein receptor, such as farnesoid X receptor. Upon binding of an agonist, farnesoid X receptor translocates from the cytoplasm to the nucleus. Microscopic techniques can be used to visualize and quantitate the amount of farnesoid X receptor located in the nucleus. In some embodiments, this assay can utilize a chimeric farnesoid X receptor fused to a fluorescent protein.

An agent can also be evaluated for its ability to increase or decrease the expression of genes known to be modulated by the farnesoid X receptor in vivo, using Northern-blot, RT PCR or oligonucleotide microarray analysis to analyze RNA levels. Western-blot analysis can be used to measure expression of proteins encoded by farnesoid X receptor target genes. Genes known to be regulated by the farnesoid X receptor include cholesterol 7 α-hydroxylase (CYP7A1), the rate limiting enzyme in the conversion of cholesterol to bile acids, fatty acid synthase (FAS), the small heterodimer partner (SHP), the bile salt export pump (BSEP, ABCB 11), canalicular bile acid export protein, the multiple drug resistance-2 (MDR-2, ABCB4), sodium taurocholate cotransporting polypeptide (NTCP, SLC10A1) and intestinal bile acid binding protein (I-BABP).

Expression of a farnesoid X receptor target gene can be conveniently normalized to an internal control and the data plotted as fold induction relative to untreated or vehicle treated cells. A control agent, such as an agonist, may be included along with DMSO as high and low controls respectively for normalization of the assay data.

Any agent which is a candidate for modulation of the farnesoid X receptor may be tested by the methods described above. Generally, though not necessarily, agents are tested at several different concentrations and administered one or more times to optimize the chances that activation of the receptor will be detected and recognized if present. Typically assays are performed in triplicate, for example, and vary within experimental error by less than about 15%. Each experiment is typically repeated about three or more times with similar results.

Provided herein are methods involving both in vitro and in vivo uses of an agent that modulates farnesoid X receptor activity. Such agents will typically exhibit farnesoid X receptor agonist, partial agonist, partial antagonist or antagonist activity in one of the in vitro or in vivo assays described herein. Methods of altering farnesoid X receptor activity, by contacting the receptor with at least one agent, are provided.

In some embodiments, the effects of agents and compositions on farnesoid X receptor gene expression and activity can be evaluated in vitro or in vivo. In some embodiments, a FXR modulator is identified in in vitro and in vivo assays. After the treatment with agents on cell lines, factors and effects directly regulated or indirectly regulated by FXR can be monitored. For example, the level of CYP27B1, 1α,25-dihydroxyvitamin D₃, and VDR activity may be measured.

After the administration of agents to animals, various tissues can be harvested to measure the effect of agents on factors or features directly or indirectly regulated by farnesoid X receptor. The level of CYP27B1, 1α,25-dihydroxyvitamin D₃, and VDR activity may be measured. In addition, levels of MMPs, parathyroid hormone, extracellular matrix proteins, serum creatinine, serum albumin, and RAS pathway can be measured. In some embodiments, the levels of mRNA are measured with Northern blot, RT-PCR, or oligonucleotide microarray analysis. In some embodiments, protein levels are measured with Western blot or Enzyme linked immunosorbent assay (ELISA). In some embodiments, the activities of at least one of CYP27B1, 1α,25-dihydroxyvitamin D₃, VDR, MMPs, parathyroid hormone, and RAS pathway, are evaluated. In some embodiments, the effects of an FXR agonist on at least one of proteinuria, lipid metabolism, renal lipid deposition, mesangial expansion, glomerulosclerosis, kidney inflammation, blood pressure, bone resorption, and bone formation are evaluated. In some embodiments, the effects of an FXR agonist on at least one condition that can be treated by elevating the VDR activity level in a patient are determined. In some embodiments, the effects of an FXR agonist on at least one disease characterized by deficient VDR activity levels in the patient are determined. In some embodiments, the effects of an FXR agonist on at least one condition selected from obesity, glucose intolerance, diabetes, metabolic syndrome, chronic kidney disease, cardiovascular disease, and bone disease are determined.

Treatment with a FXR modulator may be associated with side effects. Provided herein is method of treating at least one condition that can be treated by elevating the vitamin receptor level with an agent selected to have fewer side effects based on its profile and activities in assays testing for farnesoid X receptor activity.

The invention is further illustrated by the following non-limiting examples.

### EXAMPLES

### Example 1

The KKAy mice are diabetic mice characterized by hyperglycemia and insulin resistance and are renally impaired with nodular glomerulosclerosis, mesangial proliferation, and abuminuria that increases with age or with a high cholesterol diet. Abnormal lipid metabolism and renal lipid accumulation may contribute to diabetic nephropathy. Administration of FXR agonist, Compound A (isopropyl 3-(3,4-difluorobenzoyl)-1,1-dimethyl-1,2,3,6-tetrahydroazepino[4,5-b]indole-5-carboxylate) improves hepatic lipid metabolism and deposition in the KKAy model of diabetic nephropathy.

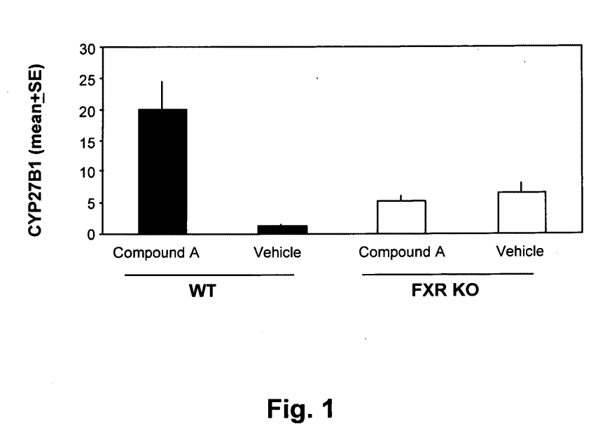

To examine the role of FXR in the kidney, gene-profiling experiments were performed using kidneys isolated from KKAy mice treated with Compound A. Genes involved in vitamin D synthesis, calcium mobilization, amino acid metabolism, inflammation, oxidative phosphorylation, and nitric oxide production were regulated by Compound A treatment. Compound A treatment upregulated CYP27B1 gene expression. The CYP27B1 gene, expressed in the proximal tubes of the kidney, encodes 25-hydroxyvitamin D₃ 1α-hydroxylase, which generates the active form of vitamin D, 1α,25-dihydroxyvitamin D₃, from a proform of vitamin D, 25 hydroxyvitamin D₃. As shown in FIG. 1, expression of CYP27B1 is induced 15-fold by 7 day treatment with Compound A in wild (WT) mice. No effect of Compound A treatment on the expression of CYP27B1 was observed in FXR deficient (FXR KO) mice indicating the FXR dependence of Compound A treatment.

### Example 2

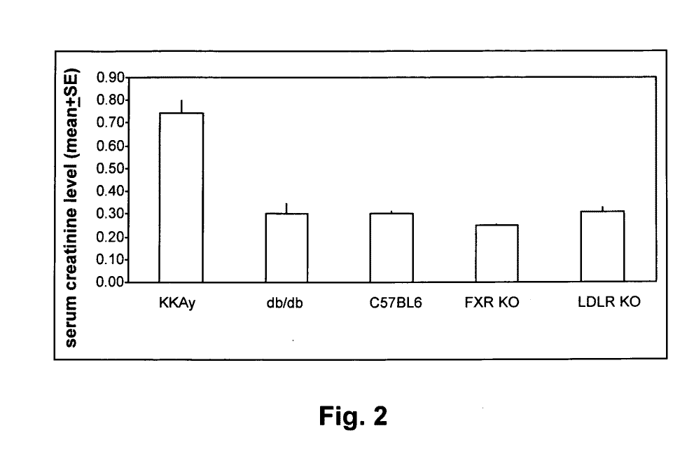

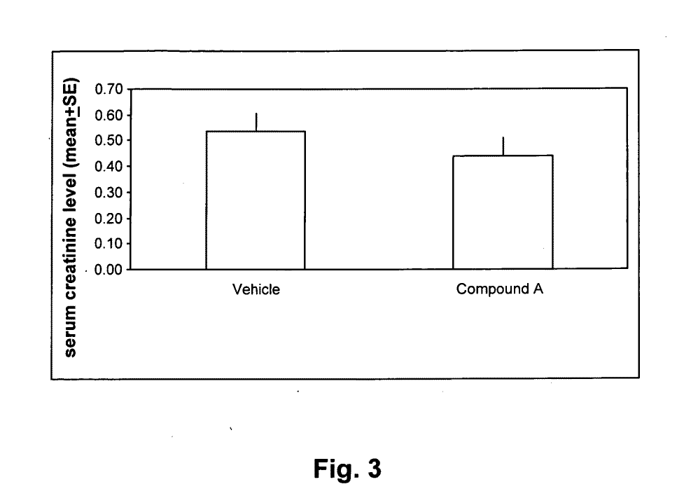

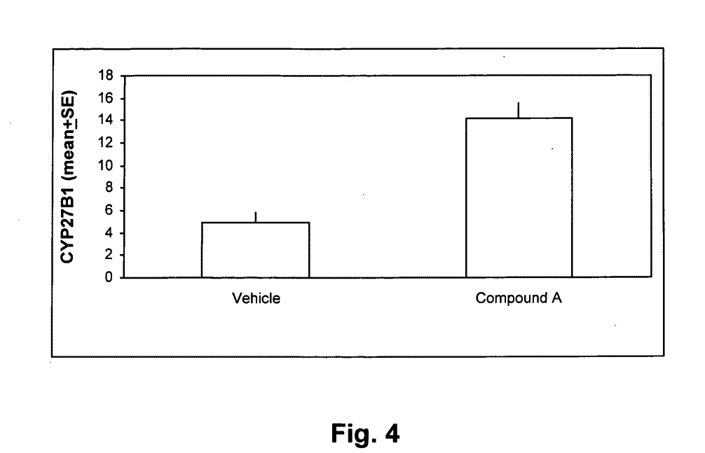

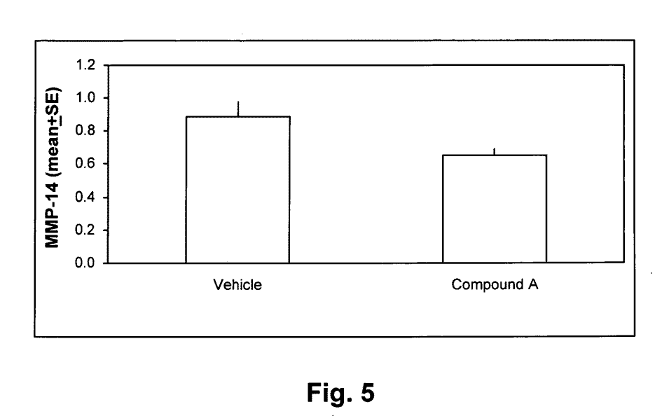

In the KKAy mice, serum creatinine levels are elevated 2.5-fold compared to db/db, C57B1/6, FXR KO, and LDLR KO mice, suggesting that KKAy mice have impaired renal function (FIG. 2). As shown in FIG. 3, administration of Compound A orally once a day for seven days caused a 20% reduction in serum creatinine levels, which was not statistically significant, but suggestive that Compound A has renal protective properties. As shown in FIGS. 4 and 5, 7 day treatment with Compound A also upregulated CYP27B1 (FIG. 4) and decreased MMP-14 (FIG. 5) gene expression which may contribute to renal protection.

### Example 3

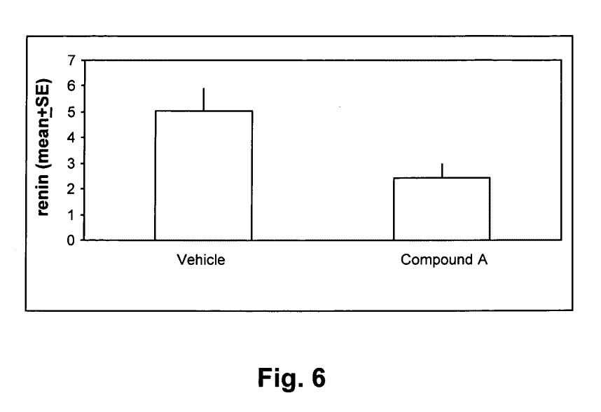

Vitamin D and its receptor vitamin D receptor (VDR) may regulate the cardiovascular system. VDR deficient and CYP27B1 deficient mice have increased blood pressure and left ventricle hypertrophy. Studies have further revealed an inverse relationship between vitamin D levels and renin activity and blood pressure in patients. Administration of compound A once a day for 4 weeks in KKAy mice caused a significant reduction in renal renin gene expression as shown in FIG. 6, indicating that compound A can affect the cardiovascular system through modulation of renin levels.

### Example 4

Vitamin D plays a critical role in bone formation as evidenced by the phenotypes described in VDR deficient and CYP27B1 deficient mice. Real time PCR gene expression data generated from Taqman® analysis indicate MC3T3 osteoblasts may express FXR. Therefore, FXR may act in paracrine and autocrine mechanisms to regulate active vitamin D production.

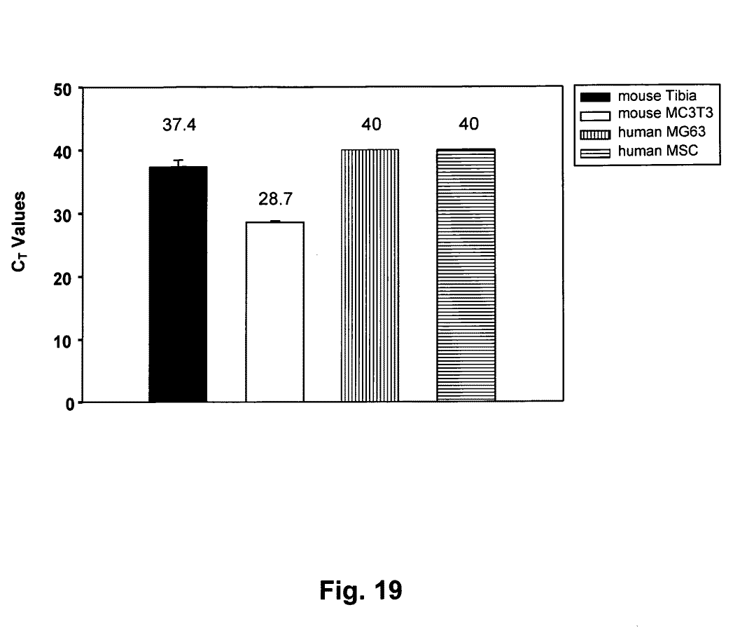

Data presented in FIG. 19 shows the expression of FXR is four cell types.

Analysis of bone mineral density in FXR deficient mice has been performed to determine if they are predisposed to osteoporosis compared to wild mice. As shown in Table 1, a significant reduction in trabecular bone density was observed in 22 week old and 28 week old, male and female, FXR deficient mice compared to wild type control mice. A significant reduction in cortical bone density was observed in 22 week old male mice compared to male wild type control mice, and a significant reduction in cortical bone density was observed in 22 and 28 week old female FXR deficient mice compared to female control wildtype mice.

TABLE 1
Evaluation of skeletal phenotype of FXR WT & KO male and female mice.

| | | | | | | | |
|---|---|---|---|---|---|---|---|
| | | Total | Trabecular | Cortical | Cortical | Periosteal | Endosteal |
| Treatment | N | Density^{a} | Density^{a} | Density^{a} | Thickness^{b} | Circ.^{b} | Circ.^{b} |

22 weeks old

| | | | | | | | |
|---|---|---|---|---|---|---|---|
| WT-Male | 7 | 524.6 ± 14.0 | 224.5 ± 12.0 | 1216.6 ± 6.2 | 0.293 ± 0.002 | 5.27 ± 0.03 | 3.43 ± 0.03 |
| KO-Male | 8 | 471.9* ± 11.3 | 177.3* ± 12.7 | 1191.0* ± 6.8 | 0.276** ± 0.004 | 5.25 ± 0.04 | 3.51 ± 0.05 |
| WT-Female | 10 | 571.9 ± 11.3 | 133.3 ± 7.0 | 1213.4 ± 4.9 | 0.288 ± 0.004 | 4.91 ± 0.05 | 3.10 ± 0.05 |
| KO-Female | 10 | 462.8** ± 12.3 | 88.00** ± 5.28 | 1154.1** ± 5.3 | 0.255** ± 0.004 | 4.61** ± 0.05 | 3.01 ± 0.05 |

28 weeks old

| | | | | | | | |
|---|---|---|---|---|---|---|---|
| WT-Male | 9 | 495.8 ± 17.3 | 199.7 ± 9.0 | 1208.0 ± 12.0 | 0.288 ± 0.006 | 5.15 ± 0.05 | 3.35 ± 0.07 |
| KO-Male | 6 | 467.0 ± 13.6 | 158.2* ± 12.2 | 1226.3 ± 2.7 | 0.274 ± 0.004 | 5.31 ± 0.06 | 3.59* ± 0.07 |
| WT-Female | 9 | 556.9 ± 16.8 | 109.8 ± 8.2 | 1230.0 ± 12.0 | 0.299 ± 0.014 | 4.96 ± 0.07 | 3.08 ± 0.03 |
| KO-Female | 8 | 478.8** ± 9.5 | 87.3* ± 4.3 | 1179.5** ± 7.1 | 0.253** ± 0.003 | 4.75* ± 0.03 | 3.16 ± 0.04 |

^{a}Mean (mg/cm³) ± SEM
^{b}Mean (mm) ± SEM
*p < 0.05 vs. corresponding WT value (Student T-Test)
**p < 0.01 vs. corresponding WT value (Student T-Test)

### Example 5

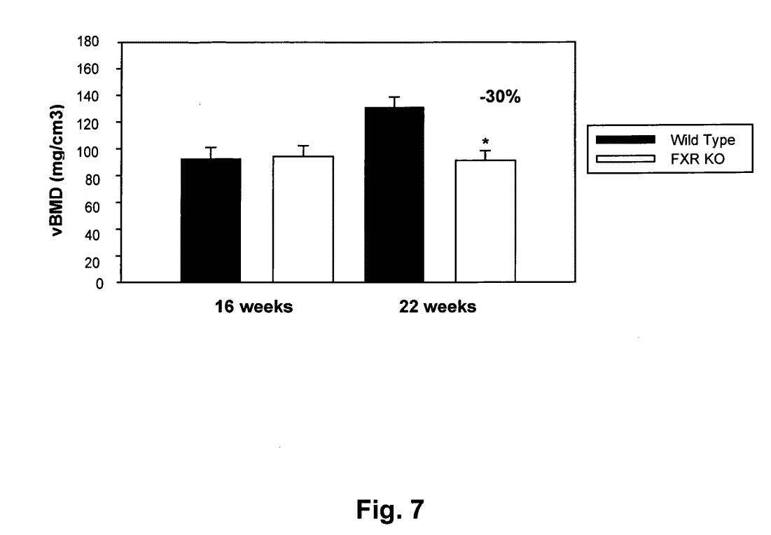

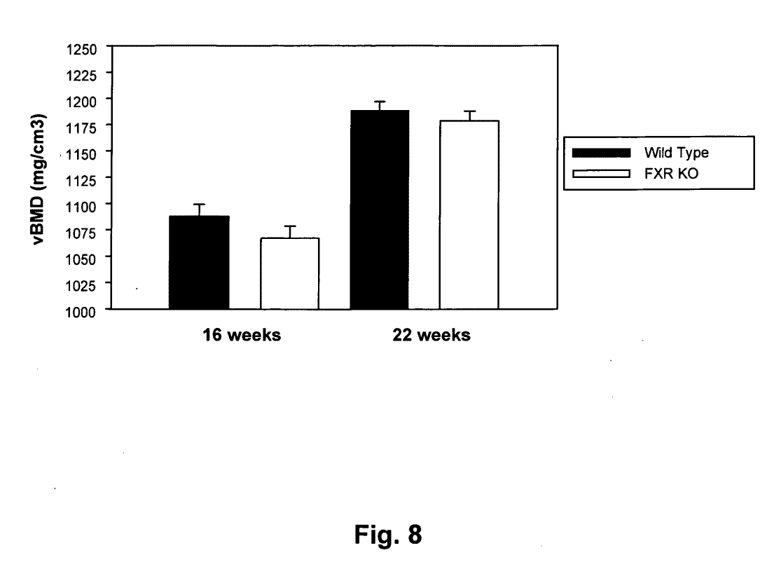

To evaluate the role of FXR in bone formation and regulation, bone mineral density (BMD) at femurs was evaluated by pQCT in FXR −/− and wild type mice at 16 and 22 weeks. As shown in FIG. 7, trabecular BMD in the FXR −/− mice was decreased by a statistically significant 30% compared to wild type mice at 22 weeks. In contrast, no difference was observed at 16 weeks. As shown in FIG. 8, no change in cortical BMD was observed between FXR −/− and wild type mice at either 16 or 22 weeks.

### Example 6

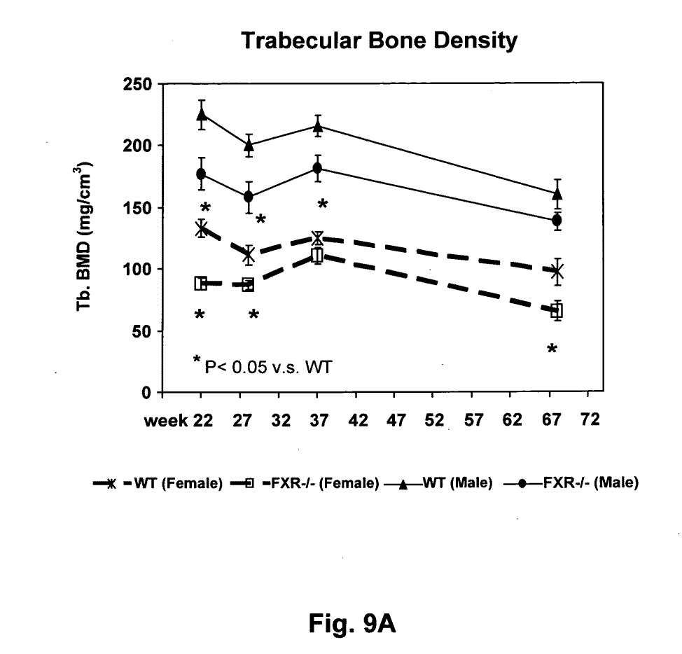

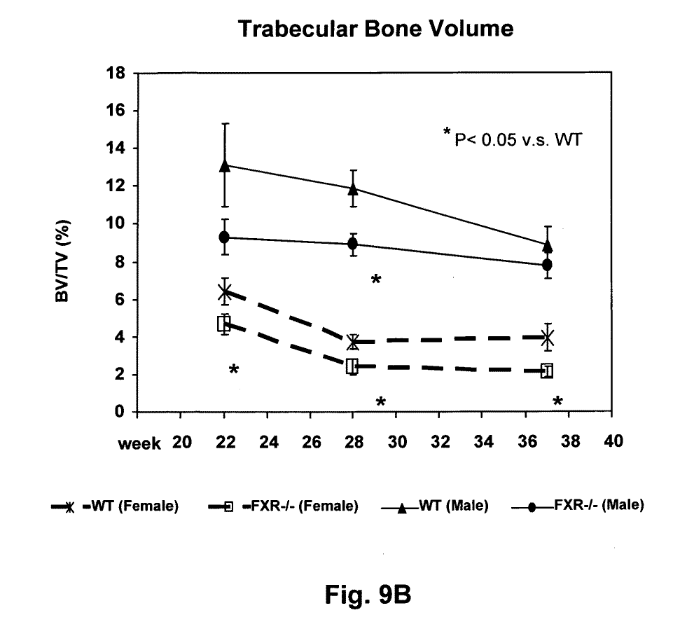

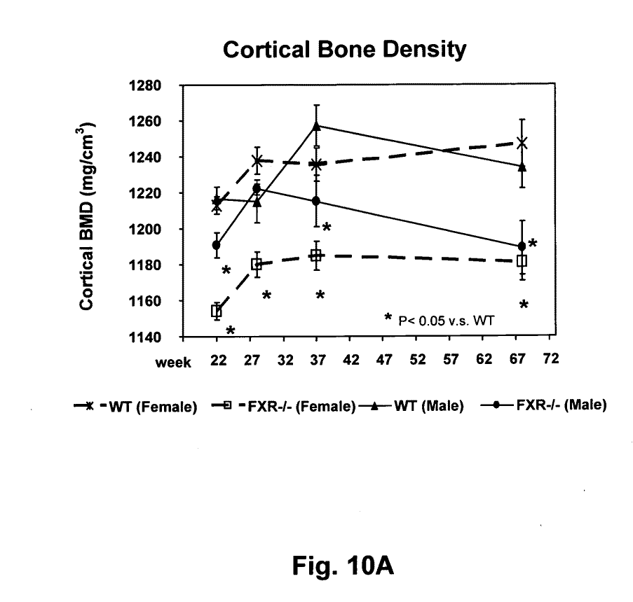

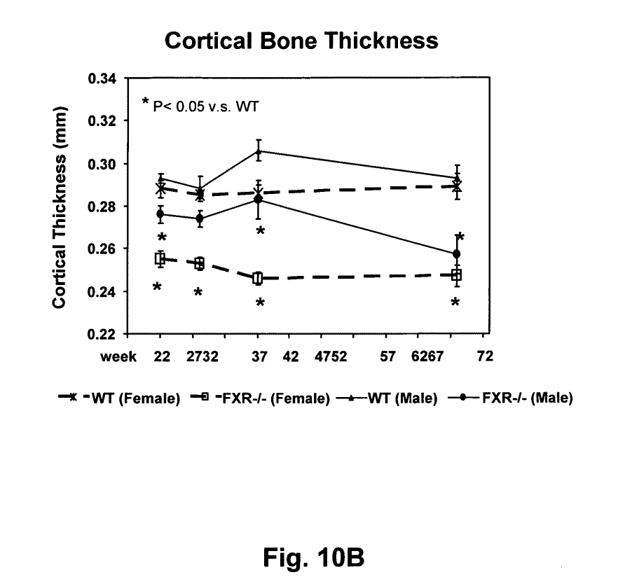

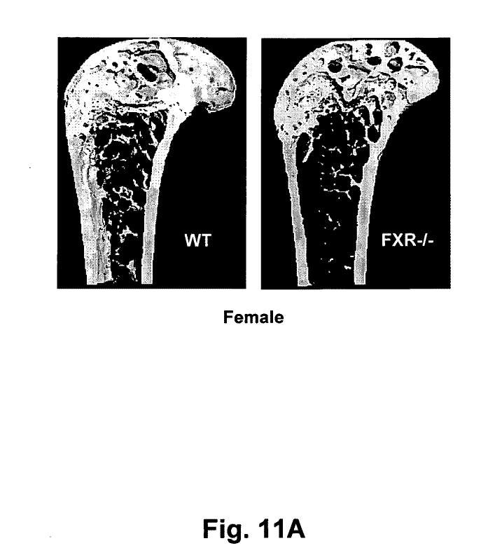

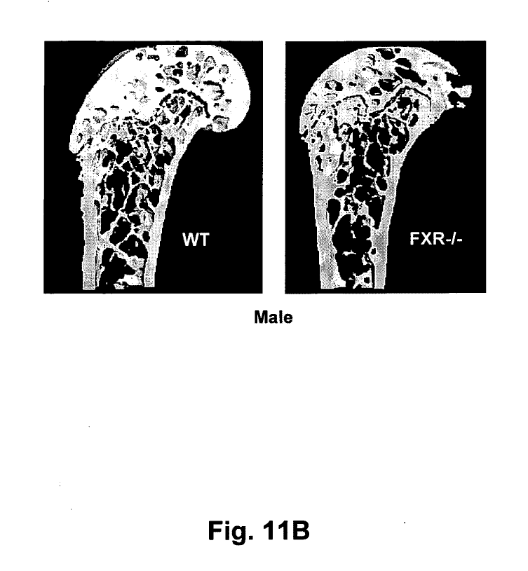

To confirm the finding presented in Example 5, femurs from both male and female FXR −/− and wild type mice were evaluated at the ages of 22, 28, 37, and 68 weeks. As shown in FIGS. 9A and 9B, a reduced trabecular BMD (pQCT) and trabecular bone volume (histomorphometric analysis) were observed at the distal femoral metaphysis in both female and male FXR −/− mice compared to wild type controls at ages between 22 and 68 weeks. As shown in FIGS. 10A and 10B, a reduced cortical BMD and thickness were observed at the femoral diaphhysis in both female and male FXR −/− mice compared to wild type controls at ages between 22 and 68 weeks. These differences are reflected in the images of distal femurs from male and female wild type and FXR −/− mice at 22 weeks, shown in FIGS. 11A and 11B. As shown in the figures, thinner trabeculae and less trabecular bone was observed in FXR −/− mice when compared to the age matched wild type control from the same gender.

### Example 7

The experimental results described above show that FXR −/− mice have less bone than wild type controls. One way this could come about is if the rate of bone formation is lower than the rate of bone formation, resulting in a negative balance. Three mechanisms that could lead to such a situation are: (1) FXR −/− mice exhibit a decrease in bone formation compared to wild type controls; (2) FXR −/− mice exhibit an increase in bone resorption compared to wild type controls; or (3) there is an increase in bone turnover in FXR −/− mice compared to controls, which leads to a situation in which the rate of bone resorption is higher than the rate of bone formation. To investigate these mechanisms in FXR −/− mice a histomorphomeric analysis was conducted at the distal femoral metaphysis in both male and female mice at age 22 weeks.

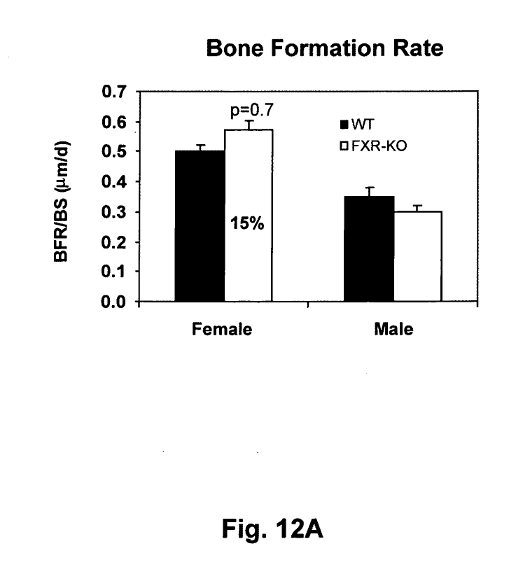

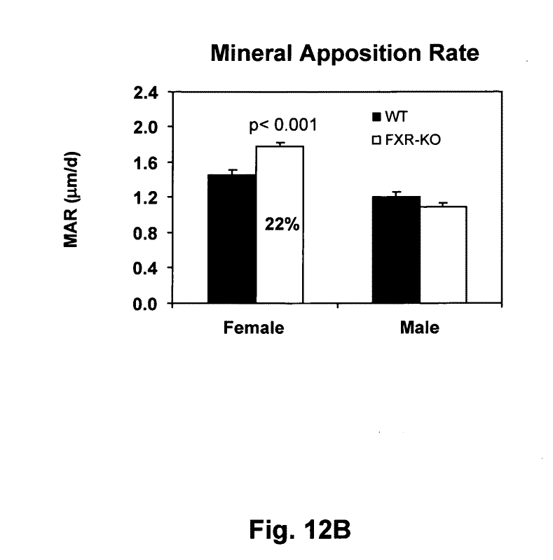

As shown in FIG. 12A, a 15% increase was observed in the bone formation rate in female FXR −/− mice compared to wild type. The mineral apposition rate is an indicator of osteoblast activity and, as shown in FIG. 12B, and 22% increase in the mineral apposition rate was observed in female FXR −/− mice compared to wild type. FIGS. 12A and 12B also indicate that no increase in either the bone formation or mineral apposition rates were observed in male FXR −/− mice compared to wild type controls, however. (In fact, and insignificant decrease in both rates was observed in male FXR −/− mice compared to wild type controls.)

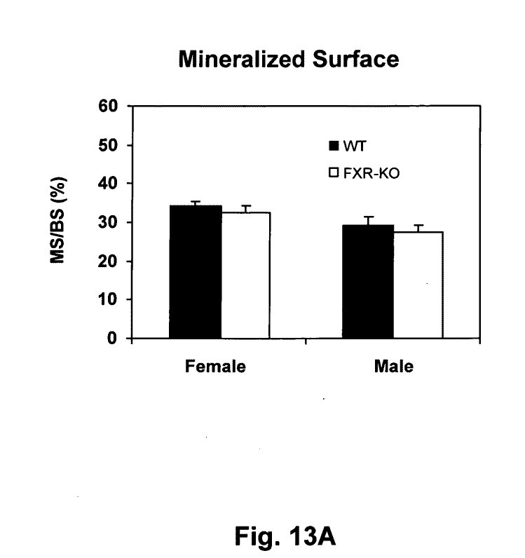

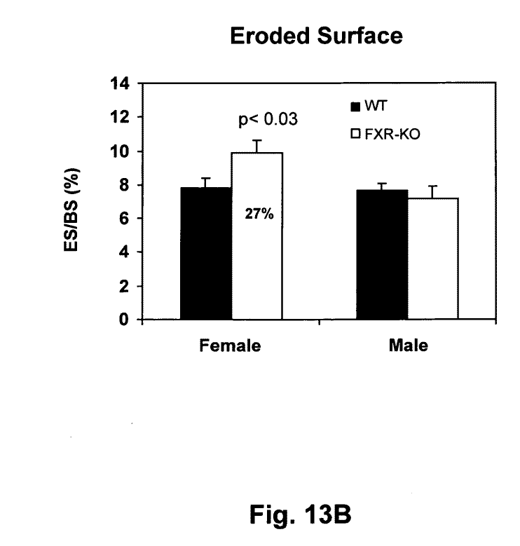

FIG. 13A shows results of an analysis of bone mineralized surface in male and female FXR −/− and wild type mice. The bone mineralized surface is an indicator of bone forming surface. As shown in FIG. 13A, no significant change was found in mineralized surface (or bone forming surface) in either male or female FXR −/− mice compared to wild type controls.

FIG. 13B shows results of an analysis of bone eroded surface male and female FXR −/− and wild type mice. The bone eroded surface is an indicator of bone resorption surface. As shown in FIG. 13B, an increase in eroded surface (or bone resorption surface) was observed in female FXR −/− mice compared to control mice, but not in male FXR −/− mice.

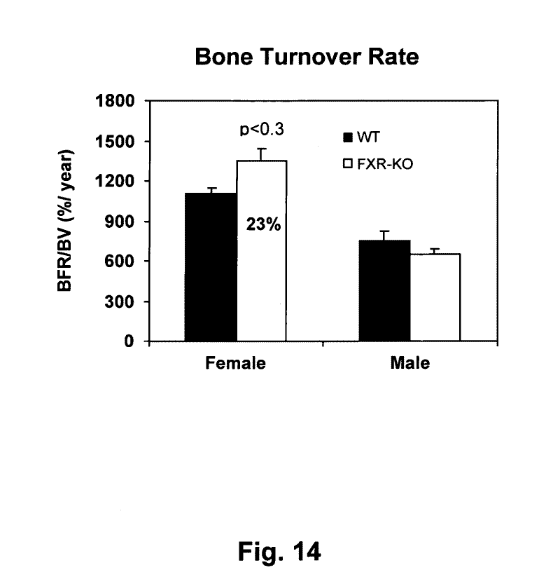

Finally, FIG. 14 presents results of an analysis of bone turnover rate. An increase in bone turnover rate indicates an increase in both bone formation and resorption. However, in most cases in which an increase in bone turnover rate occurs it leads a higher rate of bone resorption than bone formation, which in turn causes bone loss. As shown in FIG. 14, an increase in bone turnover rate was observed in female FXR −/− mice compared to control mice, but not in male FXR −/− mice.

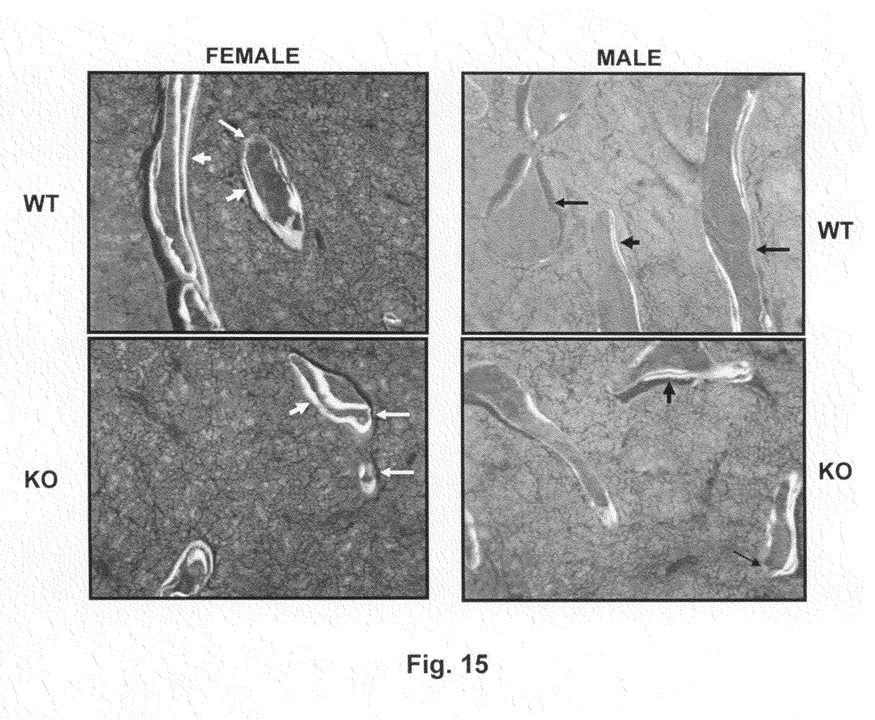

FIG. 15 shows histological images of distal femurs. These data show that deletion of FXR resulted in an osteopenic phenotype in both female and male mice, evident by lower trabecular bone density and volume as well as reduced cortical bone density and thickness. Dynamic histomorphometric data suggests that increase in bone turnover may contribute to FXR deletion-induced osteopenia in female mice, while a different mechanism of action may be involved in the osteopenic phenotype in male mice.

### Example 8

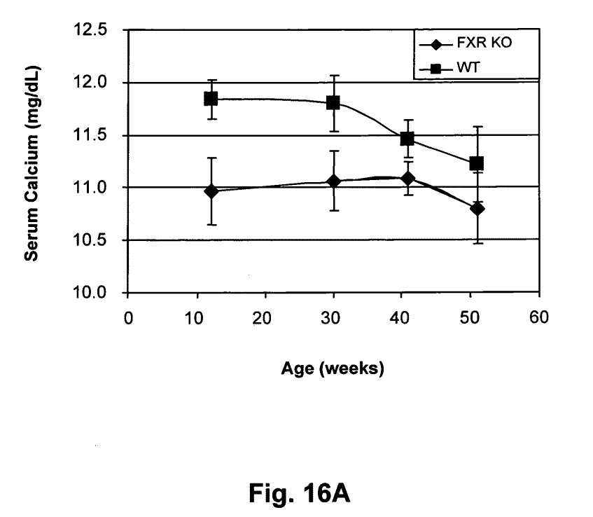

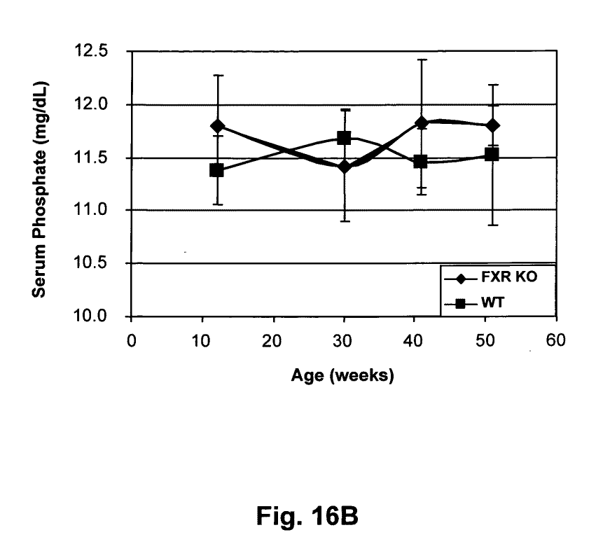

To further understand the mechanism of bone loss in FXR −/− mice, levels of serum calcium and phosphate were measured over time. As shown in FIG. 16A, FXR −/− mice have decreased levels of serum calcium through the course of their lifetime compared to wild type controls. In contrast, serum phosphate levels were not consistently different between FXR −/− and wild type controls. Decreased levels of serum calcium are consistent with a mechanism of increased bone turnover in the FXR −/− mice.

### Example 9

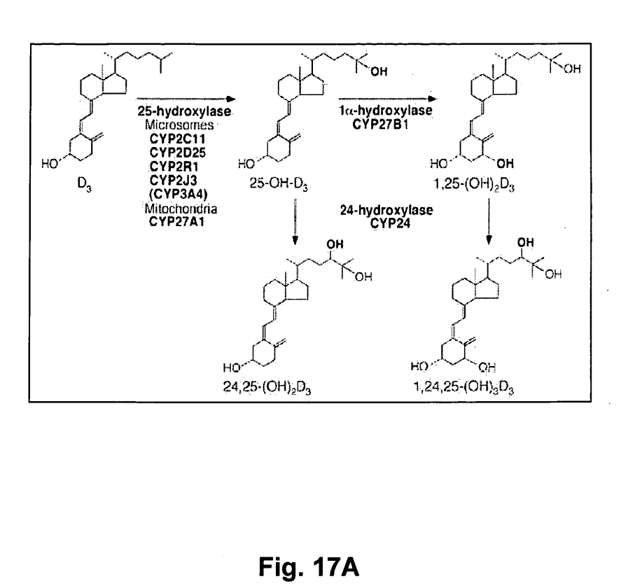

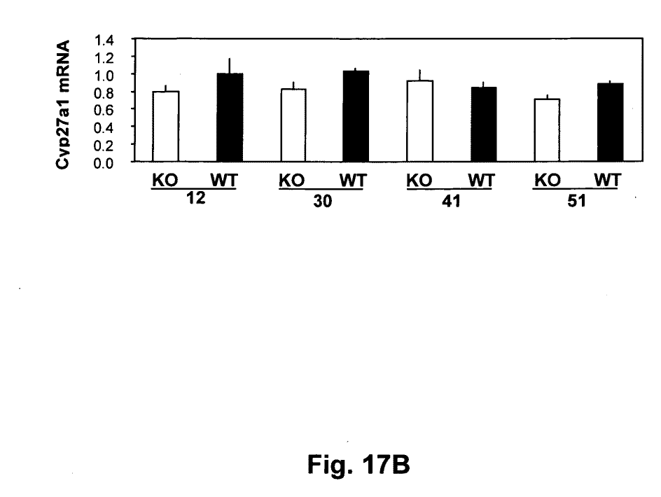

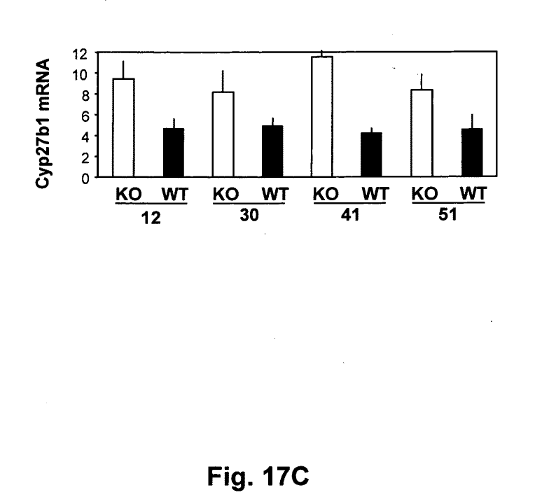

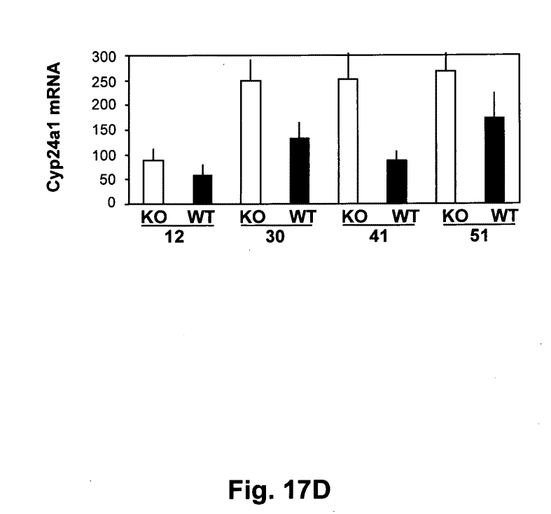

FIG. 17A shows the reactions by which vitamin D₃ is converted to 25-OH-D₃, 1,25-(OH)₂D₃, 24,25-(OH)₂D₃ and 1,24,25-(OH)₃D₃, and the roles of the Cyp27a1, Cyp27b1, and Cyp24 enzymes in those reactions. As shown in FIGS. 17B-17D, have altered expression of both renal Cyp27b1 and Cyp24a 1.

### Example 9

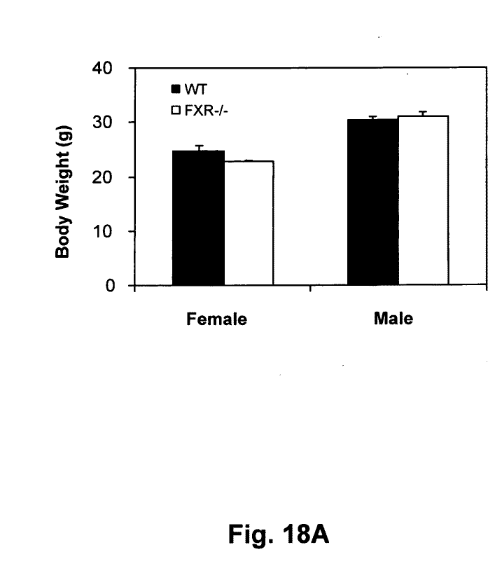

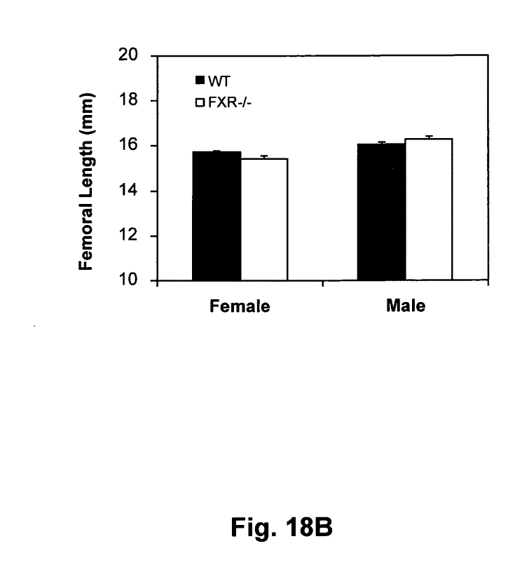

Body weight and femoral length was determined in male and female wild type and FXR −/− mice at 22, 28, 37, and 68 weeks of age. As shown in FIGS. 18A and 18B, no significant difference was observed at 22 weeks in FXR −/− mice when compared to the age matches wild type control mice of the same gender. Similar results were also observed at 28, 37, and 68 weeks.

### Example 10

OVX rats are rats subjected to bilateral ovariectomy, which results in development of osteopenia. Two studies have been conducted to evaluate the effect of one of the FXR agonists disclosed herein, isopropyl 3-(3,4-difluorobenzoyl)-1,1-dimethyl-1,2,3,6-tetrahydroazepino[4,5-b]-indole-5-carboxylate, in OVX rats. The compound at 3 and 30 mg/kg did not show any effect on preventing or restoring OVX-induced bone loss after 6-10 weeks of treatment. The exposure level of the compound in bone tissue of the rats was not studied and the EC₅₀ of the compound in bone cells was not studied. Accordingly, the reason for the absence of an observable effect has not been determined.

While some embodiments have been shown and described, various modifications and substitutions may be made thereto without departing from the spirit and scope of the invention. For example, for claim construction purposes, it is not the literal language thereof, and it is thus not intended that exemplary embodiments from the specification be read into the claims. Accordingly, it is to be understood that the present invention has been described by way of illustration and not limitations on the scope of the claims.

1. A method of treating at least one condition that can be treated by elevating the vitamin D receptor (VDR) activity level in a patient, the method comprising administering to the patient a therapeutically effective amount of at least one farnesoid X receptor (FXR) agonist, wherein the at least one FXR agonist elevates the level of Cytochrome P450, family 27, subfamily B, polypeptide 1 (CYP27B1), to thereby elevate the level of VDR activity in the patient.

2. The method of claim 1, wherein the at least one condition is a disease characterized by deficient VDR activity levels in the patient.

3. The method of claim 1, wherein the level of CYP27B1 is elevated in at least one cell type of the patient selected from kidney cells and bone cells.

4. The method of claim 3, wherein the level of CYP27B1 is elevated in at least one bone cell type of the patient selected from osteoblasts and osteoclasts.

5. The method of claim 1, wherein the at least one FXR agonist elevates the level of CYP27B1, to thereby elevate the level of 1α,25-dihydroxyvitamin D₃ in at least one of serum of the patient and a cell type of the patient selected from kidney cells and bone cells.

6. (canceled)

7. The method of claim 1, wherein the VDR activity level is elevated in at least one cell type of the patient selected from kidney cells, cardiomyocytes, bone cells, immune cells, mesangial cells, and smooth muscle cells.

8-9. (canceled)

10. The method of claim 1, wherein administration of the at least one FXR agonist does not cause at least one of hypercalcemia and hypercalcinuria in the patient.

11. The method of claim 1, wherein the at least one condition is selected from obesity, glucose intolerance, diabetes, and metabolic syndrome.

12. The method of claim 1, wherein the at least one condition is chronic kidney disease.

13. (canceled)

14. The method of claim 12, wherein treatment of the chronic kidney disease comprises treatment of at least one secondary disorder in the patient selected from parahyperthyroidism and cardiovascular disease.

15. (canceled)

16. The method of claim 12, wherein the at least one FXR agonist reduces the level of at least one of a matrix metalloprotease (MMP), an extracellular matrix protein, renin angiotensin system (RAS) pathway, parathyroid hormone, serum creatinine, serum albumin, proteinuria, lipid metabolism, renal lipid deposition, mesangial expansion, glomerulosclerosis, and kidney inflammation in the patient.

17-20. (canceled)

21. The method of claim 1, wherein the at least one condition is cardiovascular disease.

22. The method of claim 21, wherein the cardiovascular disease is characterized by at least one of coronary heart disease, cerebrovascular disease, peripheral vascular disease, congestive heart failure, myocardial infarction, left ventricular hypertrophy, hypertension, and atherosclerosis.

23-29. (canceled)

30. The method of claim 1, wherein the at least one FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl) 1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoy1)-1,4,4-trimethy1-1,4,5,6-tetrahydro-pyrro1o[2,3-d]azepine-2,8-dicarboxylic acid 2-ethy1 ester 8-isopropy1 ester;
6-(3,4-difluoro-benzoy1)-4,4-dimethy1-1,4,5,6-tetrahydro-pyrro1o[2,3-d]azepine-2,8-dicarboxylic acid 2-ethy1 ester 8-isopropy1 ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro-4H-thieno[2,3-D]azepine-8-carboxylic acid ethyl ester;
6-(4-fluoro-benzoyl)-3,6,7,8-tetrahydro-imidazo[4,5-D]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3(4-fluorobenzoyl)-2-methyl-1,2,3,6-tetrahydroazepino[4,5-b]indole-5-carboxylate; and
n-propyl 3(4-fluorobenzoyl)-2-methyl-8-fluoro-1,2,3,6-tetrahydroazepino[4,5-b]indole-5-carboxylate.

31. A method of modulating the level of at least one of CYP27B1 and 1α,25-dihydroxyvitamin D₃ in a cell, comprising providing an effective amount of at least one FXR modulator, to thereby modulate the level of at least one of CYP27B1 and 1α,25-dihydroxyvitamin D₃ in the cell.

32. The method of claim 31, wherein the level of at least one of CYP27B1 and 1α,25-dihydroxyvitamin D₃ is elevated in the cell and wherein the at least one FXR modulator is a FXR agonist.

33. The method of claim 32, wherein the FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl) 1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoy1)-1,4,4-trimethy1-1,4,5,6-tetrahydro-pyrro1o[2,3-d]azepine-2,8-dicarboxylic acid 2-ethy1 ester 8-isopropy1 ester;
6-(3,4-difluoro-benzoy1)-4,4-dimethy1-1,4,5,6-tetrahydro-pyrro1o[2,3-d]azepine-2,8-dicarboxylic acid 2-ethy1 ester 8-isopropy1 ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro4H-thieno[2,3-D]azepine-8-carboxylic acid ethyl ester;
6-(4-fluoro-benzoyl)-3,6,7,8-tetrahydro-imidazo[4,5-D]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3(4-fluorobenzoyl)-2-methyl-1,2,3,6-tetrahydroazepino[4,5-b]indole-5-carboxylate; and
n-propyl 3(4-fluorobenzoyl)-2-methyl-8-fluoro-1,2,3,6-tetrahydroazepino[4,5-b]indole-5-carboxylate.

34. A method of modulating the VDR activity level in a patient, comprising administering to the patient an effective amount of at least one FXR modulator, to thereby modulate the VDR activity level in the patient.

35. The method of claim 34, wherein the VDR activity level is elevated in the patient and wherein the at least one FXR modulator is a FXR agonist.

36. The method of claim 35, wherein the FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl) 1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoy1)-1,4,4-trimethy1-1,4,5,6-tetrahydro-pyrro1o[2,3-d]azepine-2,8-dicarboxylic acid 2-ethy1 ester 8-isopropy1 ester;
6-(3,4-difluoro-benzoy1)-4,4-dimethy1-1,4,5,6-tetrahydro-pyrro1o[2,3-d]azepine-2,8-dicarboxylic acid 2-ethy1 ester 8-isopropy1 ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro4H-thieno[2,3-D]azepine-8-carboxylic acid ethyl ester;
6-(4-fluoro-benzoyl)-3,6,7,8-tetrahydro-imidazo[4,5-D]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3(4-fluorobenzoyl)-2-methyl-1,2,3,6-tetrahydroazepino[4,5-b]indole-5-carboxylate; and
n-propyl 3(4-fluorobenzoyl)-2-methyl-8-fluoro-1,2,3,6-tetrahydroazepino[4,5-b]indole-5-carboxylate.

37-66. (canceled)
